(19) United States
(12) Patent Application Publication
(10) Pub. No.: US 2022/0218317 A1
(43) Pub. Date: Jul. 14, 2022

(54) Tissue Removal Devices and Kits Useful for Performing Treatment under Magnetic Resonance Imaging and Related Methods

(71) Applicant: Cook Medical Technologies LLC, Bloomington, IN (US)

(72) Inventors: Neal Fearnot, West Lafayette, IN (US); Gary L. Neff, Bloomington, IN (US); Eric Brandner, West Lafayette, IN (US); Sean D. Chambers, Bloomington, IN (US); Ram H. Paul, JR., Bloomington, IN (US); Creasy Clauser Huntsman, Lafayette, IN (US)

(73) Assignee: Cook Medical Technologies LLC, Bloomington, IN (US)

(21) Appl. No.: 17/573,189

(22) Filed: Jan. 11, 2022

Related U.S. Application Data
(60) Provisional application No. 63/135,935, filed on Jan. 11, 2021.

(51) Int. Cl. A61B 10/02; A61B 5/055

(52) U.S. Cl. CPC A61B 10/0233; A61B 2090/374; A61B 5/055

(57) ABSTRACT

Example tissue removal devices and kits useful in performing treatment under magnetic resonance imaging and related methods are described. An example tissue removal device includes an elongate member and a tubular member partially disposed over the elongate member. The elongate member has a lengthwise axis and a main body that defines a lumen, a proximal portion, a coil member, and a sharp edge. The coil member extends around a portion of the lengthwise axis in a first direction. The coil member extends from the away from the lengthwise axis and away from the proximal portion. The tubular member has a proximal end, a distal end, and a main body that defines a lumen and a slot. The slot extends from the distal end of the tubular member toward the proximal end of the tubular member. The slot is sized to receive a portion of the coil member.

### RELATED APPLICATIONS

This application claims priority to U.S. Provisional Application No. 63/135,935, filed Jan. 11, 2021. The entire contents of this related application are hereby incorporated by reference into this disclosure.

### FIELD

The disclosure relates generally to the field of medical devices. More particularly, the disclosure relates to tissue removal devices useful in performing treatment under magnetic resonance imaging (MRI), kits useful in performing treatment under MRI, and methods of performing interventional medical treatment under MRI.

### BACKGROUND

The field of interventional MRI is gaining wider acceptance and seeing an increase in the number of procedures that can be performed. Interventional procedures conducted under MRI have several benefits over X-Ray-guided interventions. For example, the patient is not exposed to ionizing radiation. Also, MRI provides the ability to characterize tissue and functional flow during an interventional procedure.

The development of interventional procedures conducted under MRI has been limited as a result of the tools needed to perform these procedures being unavailable. Therefore, patients are required to make multiple visits to treatment facilities to visualize, diagnose, and treat various conditions. In addition, multiple imaging modalities are often needed, which impacts the accuracy of utilizing a magnetic resonance image in directing intervention. For example, when addressing prostate cancer, visualization, biopsy, and treatment are currently completed over the course of three patient visits. At a first visit, a scan is completed using a magnetic resonance scanner to produce an image showing the prostate and any abnormalities. The patient then leaves the facility and awaits a review of the image. If abnormalities exist, a second patient visit will occur such that a biopsy sample of the abnormal tissue can be completed. Currently, software is used to fuse the magnetic resonance image with the procedural ultrasound to provide guidance in conducting the biopsy. This fusion decreases the value of the diagnostic magnetic resonance image. The patient then leaves the facility again and awaits a review of the biopsy sample to determine whether further treatment is required (e.g., if the review results in a positive prostate cancer diagnosis). If further treatment is required, the patient will visit the facility a third time such that treatment can be performed. Completion of these three patient visits can take months, prevents the patient from receiving rapid treatment, and increases the overall costs associated with treatment. Furthermore, software used to fuse magnetic resonance images with other images (e.g., those obtained via ultrasound) have drawbacks, such as potential image overlay issues and the potential for compression shifting of tissues (e.g., prostate).

A need exists, therefore, for new and improved tissue removal devices useful in performing treatment under MRI, kits useful in performing treatment under MRI, and methods of performing interventional medical treatment under MRI.

### SUMMARY OF SELECTED EXAMPLE EMBODIMENTS

Various example tissue removal devices useful in performing treatment under MRI, kits useful in performing treatment under MRI, and methods of performing interventional medical treatment under MRI are described herein.

An example tissue removal device useful in performing treatment under MRI includes an elongate member and a tubular member partially disposed over the elongate member. The elongate member has a lengthwise axis and a main body that defines a lumen, a proximal portion, a coil member, and a sharp edge. The coil member has a first end and a second end. The first end is attached to the proximal portion. The second end is free of attachment to the proximal portion. The coil member extends from the first end around a portion of the lengthwise axis in a first direction to the second end. The coil member extends from the first end and away from the lengthwise axis to the second end. The coil member extends away from the first end and away from proximal portion to the second end. The sharp edge is defined on the second end of the coil member. The tubular member has a proximal end, a distal end, and a main body that defines a lumen and a slot. The lumen of the tubular member extends from the proximal end of the tubular member to the distal end of the tubular member. The slot extends from the distal end of the tubular member toward the proximal end of the tubular member. The slot is sized to receive a portion of the coil member.

An example kit useful in performing treatment under MRI includes a tissue removal device and a system for use with a tissue removal device.

An example method of performing an interventional medical treatment under MRI comprises: positioning a patient within a magnetic resonance scanner; scanning a first portion of the patient using the magnetic resonance scanner; obtaining a magnetic resonance image of the first portion of the patient; identifying a tissue that has predefined characteristics using the magnetic resonance image; while the patient remains positioned within the magnetic resonance scanner used to scan a portion of the patient, advancing a medical device into a bodily passage and to the tissue while scanning a second portion of the patient that includes the medical device using the magnetic resonance scanner; comprises obtaining a magnetic resonance image of the second portion of the patient that includes the medical device; confirming the position of the medical device within the bodily passage; advancing a biopsy device through the medical device and to the tissue while scanning a third portion of the patient that includes the biopsy device using the magnetic resonance scanner; obtaining a magnetic resonance image of the third portion of the patient that includes the biopsy device; confirming the position of the biopsy device; collecting a tissue sample from the tissue using the biopsy device while scanning a fourth portion of the patient that includes the biopsy device and the tissue using the magnetic resonance scanner; obtaining a magnetic resonance image of the fourth portion of the patient that includes the biopsy device; confirming the tissue sample has been collected; withdrawing the biopsy device and the tissue sample through the medical device; determining whether the tissue sample meets a predefined criterion; if the tissue sample does not meet the predefined criterion, withdrawing the medical device from the bodily passage and removing the patient from the magnetic resonance scanner; if the tissue sample meets the predefined criterion, advancing an anchor member through the medical device through which the biopsy device was advanced and to the tissue while the patient remains positioned within the magnetic resonance scanner; securing the anchor member to the tissue to retain the position of the medical device relative to the tissue; advancing a first inner sheath over the medical device and toward the tissue to dilate the bodily passage; advancing a second inner sheath over the first inner sheath and toward the tissue to dilate the bodily passage; advancing an outer sheath over the second inner sheath and toward the tissue to dilate the bodily passage; removing the anchor member from the tissue; withdrawing the anchor member from the bodily passage; withdrawing the medical device from the bodily passage; withdrawing the first inner sheath from the bodily passage; withdrawing the second inner sheath from the bodily passage; advancing a treatment device through the outer sheath and to the tissue; manipulating the tissue using the treatment device; withdrawing the treatment device from the outer sheath; scanning a first portion of the patient using the magnetic resonance scanner; advancing a tissue removal device through the outer sheath and to the tissue; manipulating the tissue using the tissue removal device; withdrawing the tissue removal device from the outer sheath; withdrawing the outer sheath from the bodily passage.

Another example method of performing an interventional medical treatment under MRI comprises: positioning a patient within a magnetic resonance scanner; scanning a first portion of the patient using the magnetic resonance scanner; obtaining a magnetic resonance image of the first portion of the patient; identifying a tissue that has predefined characteristics using the magnetic resonance image; while the patient remains positioned within the magnetic resonance scanner used to scan a portion of the patient, advancing a medical device into a bodily passage and to the tissue while scanning a second portion of the patient that includes the medical device using the magnetic resonance scanner; obtaining a magnetic resonance image of the second portion of the patient that includes the medical device; confirming the position of the medical device within the bodily passage; advancing an anchor member through the medical device and to the tissue while the patient remains positioned within the magnetic resonance scanner; securing the anchor member to the tissue to retain the position of the anchor member relative to the tissue; withdrawing the medical device from the bodily passage; advancing a first inner sheath over the anchor member and toward the tissue to dilate the bodily passage; advancing a second inner sheath over the first inner sheath and toward the tissue to dilate the bodily passage; advancing an outer sheath over the second inner sheath and toward the tissue to dilate the bodily passage; withdrawing the first inner sheath from the bodily passage; withdrawing the second inner sheath from the bodily passage; removing the anchor member from the tissue; withdrawing the anchor member from the bodily passage; advancing a biopsy device through the outer sheath and to the tissue while scanning a third portion of the patient that includes the biopsy device using the magnetic resonance scanner; obtaining a magnetic resonance image of the third portion of the patient that includes the biopsy device; confirming the position of the biopsy device; collecting a tissue sample from the tissue using the biopsy device while scanning a fourth portion of the patient that includes the biopsy device and the tissue using the magnetic resonance scanner; obtaining a magnetic resonance image of the fourth portion of the patient that includes the biopsy device; confirming the tissue sample has been collected; withdrawing the biopsy device and the tissue sample through the outer sheath; determining whether the tissue sample meets a predefined criterion; if the tissue sample does not meet the predefined criterion, withdrawing the outer sheath from the bodily passage and removing the patient from the magnetic resonance scanner; if the tissue sample meets the predefined criterion, advancing a treatment device through the outer sheath and to the tissue; manipulating the tissue using the treatment device; withdrawing the treatment device from the outer sheath; scanning a first portion of the patient using the magnetic resonance scanner; advancing a tissue removal device through the outer sheath and to the tissue; manipulating the tissue using the tissue removal device; withdrawing the tissue removal device from the outer sheath; withdrawing the outer sheath from the bodily passage; removing the patient from the magnetic resonance scanner.

An example method of performing treatment on a prostate under MRI comprises: positioning a patient within a magnetic resonance scanner; scanning a prostate and surrounding tissue of the patient using the magnetic resonance scanner; obtaining a magnetic resonance image of the prostate and surrounding tissue of the patient; identifying a tissue within the magnetic resonance image that has predefined characteristics; while the patient remains positioned within the magnetic resonance scanner used to scan the prostate and surrounding tissue, advancing a medical device into a bodily passage and to the tissue while scanning a first portion of the patient that includes the medical device using the magnetic resonance scanner; obtaining a magnetic resonance image of the second portion of the patient that includes the medical device; confirming the position of the medical device within the bodily passage; advancing a biopsy device through the medical device and to the tissue while scanning a third portion of the patient that includes the biopsy device using the magnetic resonance scanner; obtaining a magnetic resonance image of the third portion of the patient that includes the biopsy device; confirming the position of the biopsy device; collecting a tissue sample from the tissue using the biopsy device while scanning a fourth portion of the patient that includes the biopsy device and the tissue using the magnetic resonance scanner; obtaining a magnetic resonance image of the fourth portion of the patient that includes the biopsy device; confirming the tissue sample has been collected; withdrawing the biopsy device and the tissue sample through the medical device; determining whether the tissue sample meets a predefined criterion; if the tissue sample does not meet the predefined criterion, withdrawing the medical device from the bodily passage and removing the patient from the magnetic resonance scanner; if the tissue sample meets the predefined criterion, advancing an anchor member through the medical device through which the biopsy device was advanced and to the tissue while the patient remains positioned within the magnetic resonance scanner; securing the anchor member to the tissue to retain the position of the medical device relative to the tissue; advancing a first inner sheath over the medical device and toward the tissue to dilate the bodily passage; advancing a second inner sheath over the first inner sheath and toward the tissue to dilate the bodily passage; advancing an outer sheath over the second inner sheath and toward the tissue to dilate the bodily passage; removing the anchor member from the tissue; withdrawing the anchor member from the bodily passage; withdrawing the medical device from the bodily passage; withdrawing the first inner sheath from the bodily passage; withdrawing the second inner sheath from the bodily passage; advancing a treatment device through the outer sheath and to the tissue; manipulating the tissue using the treatment device; withdrawing the treatment device from the outer sheath; advancing a tissue removal device through the outer sheath and to the tissue; manipulating the tissue using the tissue removal device; withdrawing the tissue removal device from the outer sheath; withdrawing the outer sheath from the bodily passage.

Additional understanding of these example tissue removal devices, kits, and methods can be obtained by review of the detailed description, below, and the appended drawings.

### BRIEF DESCRIPTION OF THE DRAWINGS

FIG. 1 is a partial perspective view of a first example tissue removal device.

FIG. 2 is a partial perspective view of a second example tissue removal device.

FIG. 3 is a partial perspective view of a third example tissue removal device.

FIG. 4 is another partial perspective view of the tissue removal device illustrated in FIG. 3.

FIG. 5 is a top view of the tissue removal device illustrated in FIG. 3.

FIG. 6 is a partial elevation view of a fourth example tissue removal device.

FIG. 6A is a partial elevation view of an alternative tissue removal device.

FIG. 6B is a partial elevation view of another alternative tissue removal device.

FIG. 7 is a partial elevation view of a fifth example tissue removal device.

FIG. 8 is a partial elevation view of a sixth example tissue removal device.

FIG. 9 is a partial perspective view of an example system for use with a tissue removal device.

FIGS. 10A and 10B show a schematic illustration of an example method of performing treatment under MRI.

FIGS. 11A and 11B show another schematic illustration of an example method of performing treatment under MRI.

FIGS. 12A and 12B show another schematic illustration of an example method of performing treatment under MRI.

FIG. 13 illustrates an example kit that includes example tissue removal devices and an example system for use with a tissue removal device.

### DETAILED DESCRIPTION OF SELECTED EXAMPLES

The following detailed description and the appended drawings describe and illustrate various example tissue removal devices useful in performing treatment under MRI, kits useful in performing treatment under MRI, and methods of performing interventional medical treatment under MRI. The description and illustration of these examples are provided to enable one skilled in the art to make and use a tissue removal device, a kit, and to practice a method of performing an interventional medical treatment under MRI. They are not intended to limit the scope of the invention, or the protection sought, in any manner. The invention is capable of being practiced or carried out in various ways and the examples described and illustrated herein are merely selected examples of the various ways of practicing or carrying out the invention and are not considered exhaustive.

As used herein, the term “attached” refers to one member being secured to another member such that the members do not completely separate from each other during use performed in accordance with the intended use of an item that includes the members in their attached form.

As used herein, the term “circumference” refers an external enclosing boundary of a body, element, or feature and does not impart any structural configuration on the body, element, or feature.

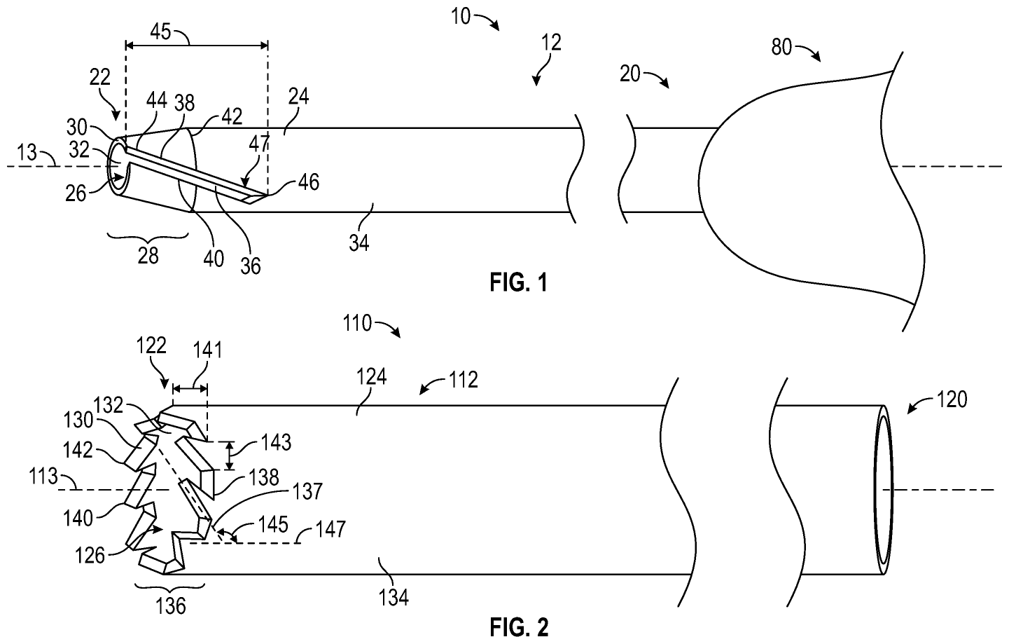

FIG. 1 illustrates a first example tissue removal device 10. In this example, the tissue removal device 10 is an elongate member 12 attached to a handle 80.

The elongate member 12 has a lengthwise axis 13, a proximal end 20, a distal end 22, and a main body 24 that defines a lumen 26, a distal tapered tip 28, a wall 30, an inner surface 32, an outer surface 34, a slot 36, a first cutting edge 38, and a second cutting edge 40. The lumen 26 extends from the proximal end 20 to the distal end 22 such that one or more devices can be passed into, and through, the elongate member 12 and/or such that the lumen 26 can be used for suction and/or flushing a portion of a bodily passage. The distal tapered tip 28 extends from a location 42 disposed between the proximal end 20 and the distal end 22 to the distal end 22 and tapers from the location 42 to the distal end 22. However, alternative embodiments can include a distal tapered portion that tapers from a location disposed between a proximal end and a distal end and toward, but not to, the distal end.

In the illustrated embodiment, the slot 36 extends from the distal end 22 toward the proximal end 20, from the inner surface 32 to the outer surface 34, around a portion of the circumference of the main body 24 and has a first end 44, a second end 46, a length 45, and a width 47. The first end 44 is disposed on the distal tapered tip 28 and the second end 46 is disposed between the distal tapered tip 28 and the proximal end 20 of the elongate member 12. The length 45 of the slot 36 extends from the first end 44 to the second end 46 and is greater than the width 47 of the slot 36. While the slot 36 has been illustrated as extending around a portion of the circumference of the main body 24, as having a first end 44 disposed on the distal tapered tip 28, as having a second end 46 disposed between the distal tapered tip 28 and the proximal end 20, and as having a length 45 that is greater than the width 47, a slot can have any suitable configuration. Selection of a suitable configuration for a slot can be based on various considerations, including the intended use of the tissue removal device. Examples of configurations considered suitable for a slot included in a tissue removal device include those in which a slot extends around the entire circumference of a main body of an elongate member, a slot extends linearly and parallel to a lengthwise axis of an elongate main body, a slot has a first end disposed between a distal tapered tip and a proximal end, a slot has a second end disposed on a distal tapered tip, a slot has a length that is less than or equal to a width of the slot, combinations of the configurations described herein, and any other configuration considered suitable for a particular embodiment.

The first cutting edge 38 extends from the first end 44 of the slot 36 to the second end 46 of the slot 36 and is defined on the outer surface 34. The second cutting edge 40 extends from the first end 44 of the slot 36 to the second end 46 of the slot 36 and is defined on the inner surface 32. This structural arrangement provides a mechanism for allowing the first cutting edge 38 to cut tissue when a torque is applied to the elongate member 12 and it is rotated in a clockwise direction and prevents additional tissue from being cut when a torque is applied to the elongate member 12 and it is rotated in a counterclockwise direction. When the elongate member 12 is rotated in a clockwise direction, tissue is cut and enters the slot 36 such that when the elongate member 12 is rotated in a counterclockwise direction the cut tissue can be separated and removed from the body. An elongate member can also include a structural arrangement in which rotation in directions opposite to those described will achieve the results described. While the cutting edges 38, 40 have been illustrated as extending from the first end 44 of the slot 36 to the second end 46 of the slot 36, a cutting edge can extend along any suitable portion of a slot. For example, a cutting edge can extend from a first end of a slot to a second end of a slot, a location between a first end and a second end of a slot to the second end of the slot, a location between a first end and a second end of a slot to the first end of the slot, or from a first location between a first end and a second end of a slot to a second location between the first location and the first end of the slot. In addition, while the first cutting edge 38 has been illustrated on the outer surface 34 and the second cutting edge 40 has been illustrated on the inner surface 32, a cutting edge can be defined on an inner surface, an outer surface, or between an inner surface and outer surface. A cutting edge defined by an elongate member can optionally be sharpened and electropolished to provide a sharp cutting surface.

The handle 80 is attached to the proximal end 20 of the elongate member 12 and is adapted to move the elongate member 12 is first and second directions. The handle 80 can include any suitable features, structures, and/or components capable of accomplishing rotation of the elongate member 12 in first and second directions. For example, rotation of an elongate member can be accomplished using a pneumatic or hydraulic actuation of gears (e.g., driven manually by the operator or otherwise). Alternately, a handle can be omitted from a tissue removal device and rotation of the tissue removal device can be accomplished manually.

In the illustrated embodiment, the elongate member 12 and handle 80 can be formed of any suitable MRI compatible material and can include any suitable type and/or number of markers. Selection of a suitable material to form an elongate member and/or handle can be based on various considerations, including the intended use of the elongate member. Examples of MRI compatible materials considered suitable to form an elongate member and/or a handle include biocompatible materials, materials that can be made biocompatible, metals, electrically insulating materials, electrically non-conducting materials, non-magnetic materials, shape memory alloys, including nickel-titanium alloys such as Nitinol, stainless steel, including Austenitic stainless steel, stainless steel containing Iron, stainless steel including Inconel, cobalt chromium, cobalt chromium alloys, Inconel, titanium, thermoplastics, polymers, PEEK, carbon-filled PEEK, ceramics, the materials described herein, combinations of the described herein, and any other material considered suitable for a particular embodiment. In the illustrated embodiment, the elongate member 12 is formed of a non-magnetic material and the handle 80 is formed of a thermoplastic. In embodiments in which a cutting or sharp edge is included in a device, a first portion of the device that extends from a proximal end toward the distal end can be formed of a first material (e.g., PEEK, Inconel) and a second portion on which the cutting or sharp edge is disposed can be attached to the first portion and formed of a second material that is different than the first material (e.g., Inconel, PEEK). The marker 50 comprises a magnetically susceptible material incorporated into the material forming the main body 14. For example, a ferromagnetic or paramagnetic compound can be incorporated into the material that forms the main body 14 during manufacture and at a desired location on the main body 14. Examples of materials considered suitable for marker or markers include magnetically susceptible material, such as ferromagnetic or paramagnetic compounds, including those in powder form, Tantalum powder, Barium Sulfate, Bismuth Oxychloride, Tungsten, Iron Oxide nanoparticles, functionalized magnetite, Gadolinium, Stainless Steel, Ferritic Stainless Steel, Ferritic Stainless Steel powders, 316 Stainless Steel, and any other material considered suitable for a particular embodiment. A marker can be incorporated into the material of the main body or disposed on a surface of the main body. Examples of markers considered suitable to include in a medical device are described in U.S. Patent Application No. 63/135,801, filed on Jan. 11, 2021, which is hereby incorporated by reference in its entirety for the purpose of describing markers considered suitable to include in a medical device. Optionally, a cutting edge, a tip, a sharp edge, a sharp cutting edge, a spiral member, and/or a barb can include one or more markers that can visualized using an image obtained by MRI or identified by a unique pattern recognized and transformed into a virtual instrument within a program and displayed on a screen.

FIG. 2 illustrates a second example tissue removal device 110. In this example, the tissue removal device 110 is an elongate member 112. The elongate member 112 is similar to the elongate member 12 illustrated in FIG. and described above, except as detailed below. In the illustrated embodiment, the elongate member 112 has a lengthwise axis 113, a proximal end 120, a distal end 122, and a main body 124 that defines a lumen 126, a wall 130, an inner surface 132, an outer surface 134, and a plurality of cutting teeth 136. The lumen 126 extends from the proximal end 120 to the distal end 122 such that one or more devices can be passed into, and through, the elongate member 112 and/or such that the lumen 126 can be used for suction and/or flushing a portion of a bodily passage.

Each tooth of the plurality of cutting teeth 136 has a lengthwise axis 137, a first end 138, a second end 140, a length 141, a tip 142, and a width 143. The first end 138 is disposed between the proximal end 120 and the distal end 122 of the elongate member 112 and the second end 140 is disposed at the distal end 122 of the elongate member 112. The length 141 extends from the first end 138 to the second end 140. The tip 142 is disposed at the distal end 122 of the elongate member 112 and defines a sharp cutting edge 144. The tip 142 of each tooth of the plurality of cutting teeth 136 can optionally be sharpened and electropolished to provide the sharp cutting edge 144. The width 143 tapers from the first end 138 to the second end 140. The lengthwise axis 137 of each tooth of the plurality of cutting teeth 136 is disposed at an angle 145 relative to an axis 147 that is parallel to the lengthwise axis 113 of the elongate member 112 and disposed within the wall 130 of the elongate member 112. The angle 145 is the same for each tooth of the plurality of cutting teeth 136. However, alternative embodiments can include a plurality of teeth of which only a portion have a lengthwise disposed at the same angle relative to an axis disposed parallel to a lengthwise axis of an elongate member. Optionally, an elongate member can include one or more markers, such as at, or near, the distal end of the elongate member, along the length of the elongate member, and any other location considered suitable for a particular embodiment.

The structural arrangement of the elongate member 112 provides a mechanism for allowing the plurality of cutting teeth 136 to cut tissue when a torque is applied to the elongate member 112 and it is rotated in a clockwise direction. When the elongate member 112 is rotated in a clockwise direction, tissue is cut and can enter the lumen 126 such that when suction is applied to the lumen 126 the cut tissue can be removed from the body. An elongate member can also include a structural arrangement in which rotation in directions opposite to those described will achieve the results described. The application of toque on the elongate member 112 to accomplish rotation can be accomplished using any suitable method or technique. In the embodiment illustrated, torque is applied manually by a user. Alternatively, a handle can be attached to an elongate member to accomplish rotation of the elongate member as described herein.

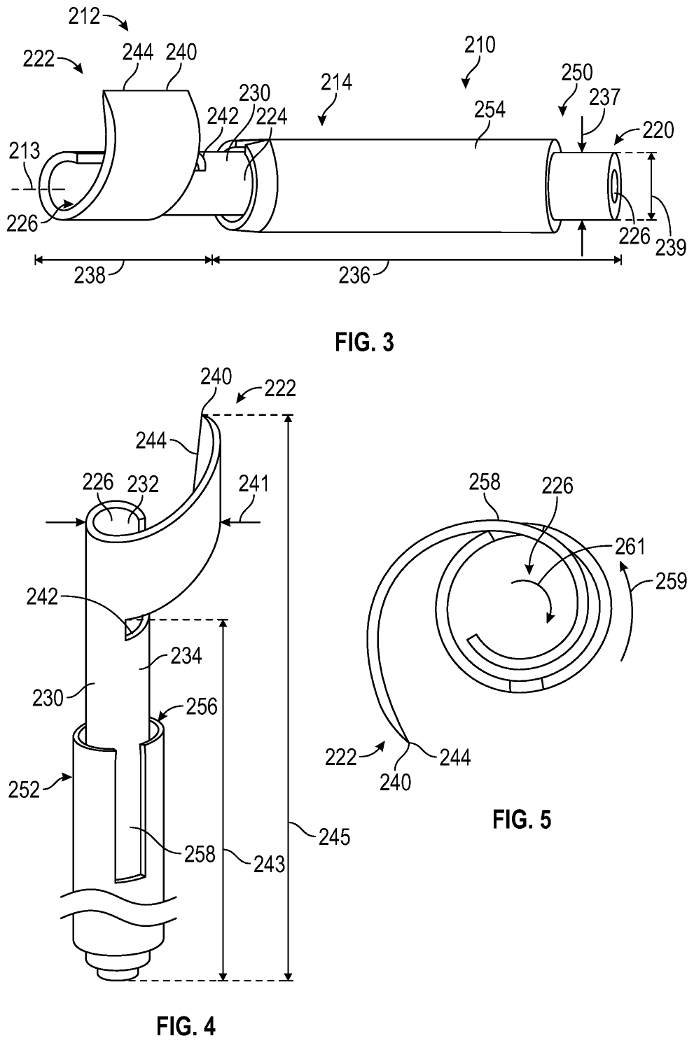

FIGS. 3, 4, and 5 illustrate a third example tissue removal device 210. In this example, the tissue removal device 210 is an elongate member 212 partially disposed within a tubular member 214. The elongate member 212 is similar to the elongate member 12 illustrated in FIG. 1 and described above, except as detailed below. In the illustrated embodiment, the elongate member 212 has a lengthwise axis 213, a proximal end 220, a distal end 222, and a main body 224 that defines a lumen 226, a wall 230, an inner surface 232, an outer surface 234, a proximal portion 236, a coil member 238, and a sharp edge 240. The lumen 226 extends from the proximal end 220 to the distal end 222 such that one or more devices can be passed into, and through, the elongate member 212 and/or such that the lumen 226 can be used for suction and/or flushing a portion of a bodily passage.

In the illustrated embodiment, the coil member 238 is moveable between a first, compressed configuration and a second, uncompressed configuration. The proximal portion 236 has an outside diameter 237 and extends from the proximal end 220 to the coil member 238. The coil member 238 has a first outside diameter 239 in the first, compressed configuration and a second outside diameter 241 in the second, uncompressed configuration that is greater than the first outside diameter 239. The coil member 238 extends from the proximal portion 236 to the distal end 222. The coil member 238 has a first end 242 and a second end 244. The first end 242 is attached to the proximal portion 236 and the second end 244 is free of attachment to the proximal portion. In the second, uncompressed configuration, the coil member 238 extends from the first end 242 around a portion of the lengthwise axis 213 of the elongate member 212 in a first direction to the second end 244. However, in alternative embodiments, a coil member can extend from a first end around the entirety of a lengthwise axis of an elongate member, or around a lengthwise axis of an elongate member more than a single revolution. In addition, in the second, uncompressed configuration, the coil member 238 extends from the first end 242 and away from the lengthwise axis 213 of the elongate member 212 to the second end 244 and from the first end 242 and away from the proximal portion 236 to the second end 244. This structural arrangement results in the coil member 238 having a second outside diameter 241 that is greater than the outside diameter 237 of the proximal portion 236. In addition, this structural arrangement results in the first end 242 being disposed a first distance 243 from the proximal end 220 of the elongate member 212 and the second end 244 being disposed a second distance 245 from the proximal end 220 of the elongate member 212 that is greater than the first distance 243. The sharp edge 240 is defined on the second end 244 of the coil member 238. The sharp edge 240 can optionally be sharpened and electropolished to provide a sharp cutting surface. Optionally, an elongate member can include one or more markers, such as at, or near, the distal end of the elongate member, along the length of the elongate member, and any other location considered suitable for a particular embodiment.

The tubular member 214 disposed over the elongate member 212 and has a proximal end 250, a distal end 252, and a main body 254 that defines a lumen 256 and a slot 258. The lumen 256 extends from the proximal end 250 to the distal end 252 and is sized to receive the elongate member 212. The slot 258 extends from the distal end 252 toward the proximal end 250 and is sized to receive a portion of the coil member 238. Optionally, a tubular member can include one or more markers, such as at, or near, the distal end of the tubular member, along the length of the tubular member, and any other location considered suitable for a particular embodiment.

In the first, compressed configuration, the coil member 238 is disposed within the lumen 256 defined by the tubular member 214. To move the coil member 238 from the first, compressed configuration to the second, uncompressed configuration, torque is applied to the elongate member 212 in a first direction, as shown by arrow 259 in FIG. 5, while the position of the tubular member 214 is maintained such that the elongate member 212 is rotated relative to the tubular member 214. Alternatively, torque can be applied to the elongate member 212 in a second direction 261 while applying torque to the tubular member 214 in a first, different direction 259, or maintaining the position of the elongate member 212 while applying torque to the elongate member 214 in a second direction 261. This results in the coil member 238 being advanced out of the lumen 256 defined by the tubular member 214 through the slot 258. Torque is applied to the elongate member 212 and/or tubular member 214, as described above until a desired amount of the coil member 238 is advanced out of the tubular member 214. Subsequently, torque can be applied to the elongate member 212 in the first direction 259 until a desired amount of tissue has been cut, or removed (e.g., by applying suction to the lumen 226), from the treatment area.

Once a desired amount of tissue has been cut, or removed, from the treatment area, the coil member 238 can be moved from the second, uncompressed configuration to the first, compressed configuration. This can be accomplished by applying torque to the elongate member 212 in a second direction 261, opposite that shown by arrow 259, while maintaining the position of the tubular member 214 such that the elongate member 212 is rotated relative to the tubular member 214. Alternatively, torque can be applied to the elongate member 212 in a first direction 259 while applying torque to the tubular member 214 in a second, different direction 261, or maintaining the position of the elongate member 212 while applying torque to the elongate member 214 in a first direction 259. This results in the coil member 238 being introduced into the lumen 256 defined by the tubular member 214 through the slot 258. Torque is applied to the elongate member 212 and/or tubular member 214, as described above until a desired amount of the coil member 238 is introduced into the tubular member 214 (e.g., a portion of the coil member 238, the entirety if the coil member 238). In embodiments in which the coil member 238 is not disposed adjacent to the slot 258 subsequent to cutting, or removing tissue, the elongate member 212 can be advanced toward the tubular member 214 by applying a proximally-directed force on the elongate member 212 while maintaining the position of the tubular member 214, applying a proximally-directed force on the elongate member 212 while applying a distally-directed force on the tubular member 214, or by maintaining the position of the elongate member 212 while applying a distally-directed force on the tubular member 214. The application of toque on the elongate member 212 to accomplish rotation can be accomplished using any suitable method or technique. In the embodiment illustrated, torque is applied manually by a user. Alternatively, a handle can be attached to an elongate member to accomplish rotation of the elongate member as described herein. Furthermore, an elongate member can include a structural arrangement in which rotation in directions opposite to those described will achieve the results described.

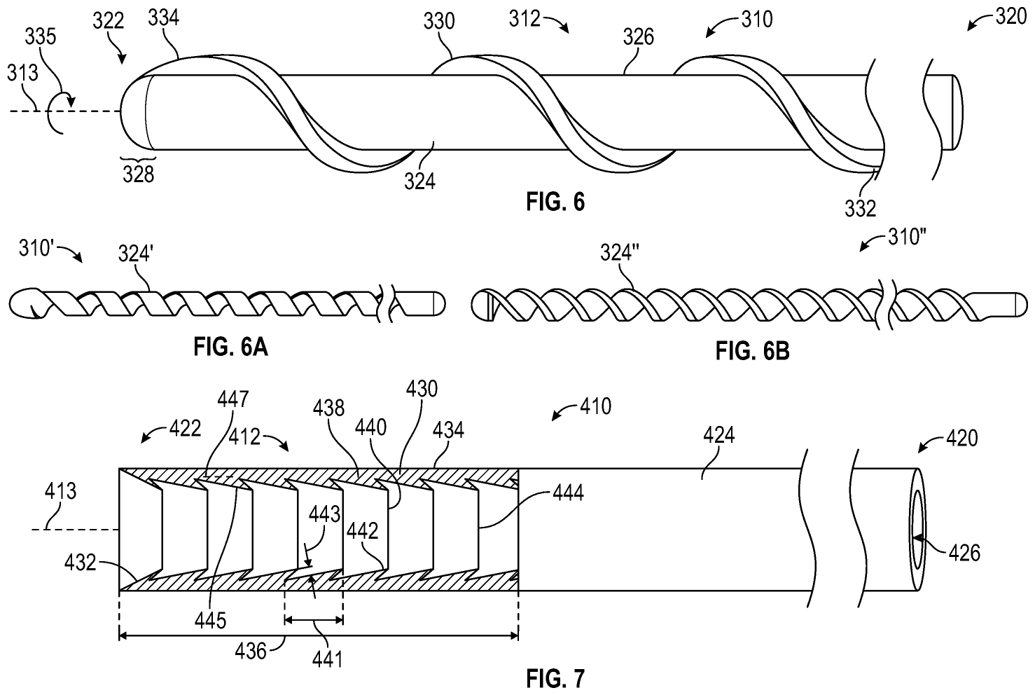

FIG. 6 illustrates a fourth example tissue removal device 310. In this example, the tissue removal device 310 is an elongate member 312. The elongate member 312 is similar to the elongate member 12 illustrated in FIG. and described above, except as detailed below. In the illustrated embodiment, the elongate member 312 has a lengthwise axis 313, a proximal end 320, a distal end 322, and a main body 324 that defines a central shaft 326, a blunted distal tip 328, and a spiral member 330. The central shaft 326 extends from the proximal end 320 to the distal end 322. The spiral member 330 extends from the central shaft 326 and away from the lengthwise axis 313 of the elongate member 312. The spiral member 330 has a first end 332, a second end 334, and extends around the lengthwise axis 313 of the elongate member 312 from the first end 332 to the second end 334. Optionally, an elongate member can include one or more markers, such as at, or near, the distal end of the elongate member, along the length of the elongate member, and any other location considered suitable for a particular embodiment. The tissue removal device 310 can include one or more markers. For example, a first marker 350 can be disposed on the proximal end 320 and a second marker 352 on the central shaft 326 at or at a location orthogonal to the first end 332 of the spiral member 330. Alternatively, a second marker 352 can be disposed on the first end 332 of the spiral member 330. Also alternatively, a marker can be disposed along the entire length of the spiral member 330. For example, the external side of the spiral member 330 can be coated with a magnetically susceptible material, such as a paramagnetic material or a ferromagnetic material, such as Nickel or a Nickel-containing alloy.

To remove tissue from a bodily passage, the elongate member 312 is positioned adjacent, or within, the tissue and a torque is applied to the elongate member 312 in a first direction, illustrated by arrow 335. Optionally, a distally-directed force can be applied to the elongate member 312 while applying a torque in the first direction 335 to remove tissue. The application of toque on the elongate member 312 to accomplish rotation can be accomplished using any suitable method or technique. In the embodiment illustrated, torque is applied manually by a user. Alternatively, a handle can be attached to an elongate member to accomplish rotation of the elongate member as described herein. Furthermore, an elongate member can include a structural arrangement in which rotation in directions opposite to those described will achieve the results described.

Each of FIGS. 6A and 6B illustrate alternative structures for the main body 324 of the fourth example tissue removal device 310. In FIG. 6A, the main body 324′ of the tissue removal device 310′ defines a ship auger, eliminating the central shaft 326 and spiral member 330 disposed on the central shaft 326 illustrated in FIG. 6. In FIG. 6B, the main body 324″ of the tissue removal device 310″ defines a twisted ribbon structure, also eliminating the central shaft 326 and spiral member 330 disposed on the central shaft 326 illustrated in FIG. 6. Each of these alternative structures may be advantageous for certain applications, and a skilled artisan will be able to select a desirable structure for a tissue removal device according to a particular embodiment based on various considerations, including the nature of the tissue with which the tissue removal device is intended to be used. Each of these alternative structures can include a marker or markers as described above for the embodiment illustrated in FIG. 6. For example, a first marker 350′ and 350″ can be disposed on the proximal end 320′ and 320″ and a second marker 352′ and 352″ on the main body 324′ 324″ at or at a location orthogonal to the first end 332′ and 332″ of the auger portion of the main body 320′ and 320″. Alternatively, a marker can be disposed along the entire length of the auger portion of the main body 320′ and 320″. For example, the external side of the auger portion of the main body 320′ and 320″ can be coated with a magnetically susceptible material, such as a paramagnetic material or a ferromagnetic material, such as Nickel or a Nickel-containing alloy.

FIG. 7 illustrate a fifth example tissue removal device 410. In this example, the tissue removal device 410 is an elongate member 412. The elongate member 412 is similar to the elongate member 12 illustrated in FIG. and described above, except as detailed below. In the illustrated embodiment, the elongate member 412 has a central lengthwise axis 413, a proximal end 420, a distal end 422, and a main body 424 that defines a lumen 426, a wall 430, an inner surface 432, an outer surface 434, and a plurality of cutting teeth 436. The lumen 426 extends from the proximal end 420 to the distal end 422 such that one or more devices or tissue can be passed into, and through, the elongate member 412 and/or such that the lumen 426 can be used for suction and/or flushing a portion of a bodily passage.

In the illustrated embodiment, each tooth of the plurality of cutting teeth 436 extends from the inner surface 432 toward the central lengthwise 413 of the elongate member 412, around the entire central lengthwise axis 413, has as a first end 438, a second end 440, a length 441, a tip 442, and a width 443. Each tooth of the plurality of cutting teeth 436 can optionally be sharpened and electropolished to provide a sharp cutting surface. Alternative embodiments, however, can include a plurality of cutting teeth in which each cutting tooth of the plurality of cutting teeth extends around a portion of a central lengthwise axis of the elongate member. The first end 438 is disposed between the outer surface 434 and the lumen 426 of the elongate member 412 and the second end 440 is disposed within the lumen 426 of the elongate member 412. The length extends from the first end 438 to the second end 440. The tip 442 is disposed at the second end 440 and defines a sharp cutting edge 444. The width 443 tapers from the first end 438 to the second end 440. Each tooth of the plurality of cutting teeth 436 is disposed at an angle 445 relative to an axis 447 that is parallel to the lengthwise axis 413 of the elongate member 412 and disposed within the wall 430 of the elongate member 412. The angle 445 is the same for each tooth of the plurality of cutting teeth 436. However, alternative embodiments can include a plurality of teeth of which only a portion have a lengthwise disposed at the same angle relative to an axis disposed parallel to a lengthwise axis of an elongate member. Optionally, an elongate member can include one or more markers, such as at, or near, the distal end of the elongate member, along the length of the elongate member, and any other location considered suitable for a particular embodiment.

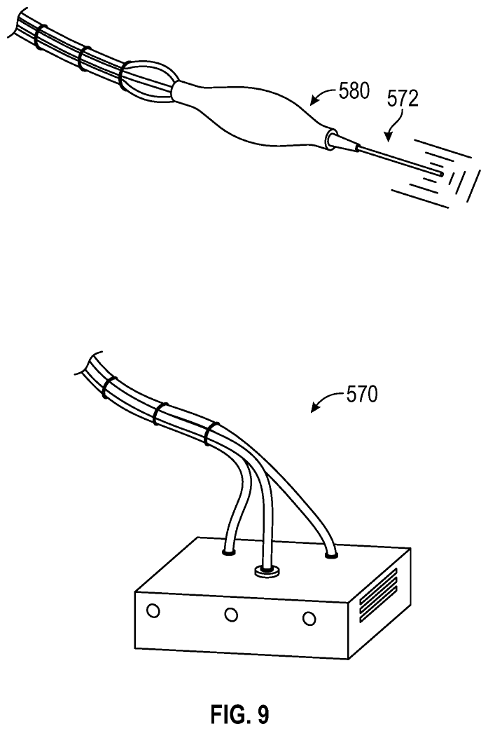

To remove tissue from a bodily passage, the elongate member 412 is positioned adjacent, or within, the tissue and an oscillating or vibrating force is applied to the elongate member 412 while applying a distally-directed force on the elongate member 412 such that tissue advances into the lumen 426 and the plurality of cutting teeth 436 mechanically disrupt the tissue. The disrupted tissue can then be removed from the bodily passage by aspiration or suction through the lumen 426 or through a lumen defined by a device through which the elongate member 412 is disposed. Alternatively, a basket and/or grasping device can be positioned distal to the elongate member 412 and through the lumen 426 or a lumen defined by a device through which the elongate member 412 is disposed and used to remove tissue. The application of an oscillating or vibrating force on the elongate member 412 can be accomplished using any suitable method or technique. In the embodiment illustrated, an oscillating or vibrating force can be applied using the system 570 illustrated in FIG. 9, as described in more detail herein. Alternatively, an oscillating or vibrating force can be applied manually by a user.

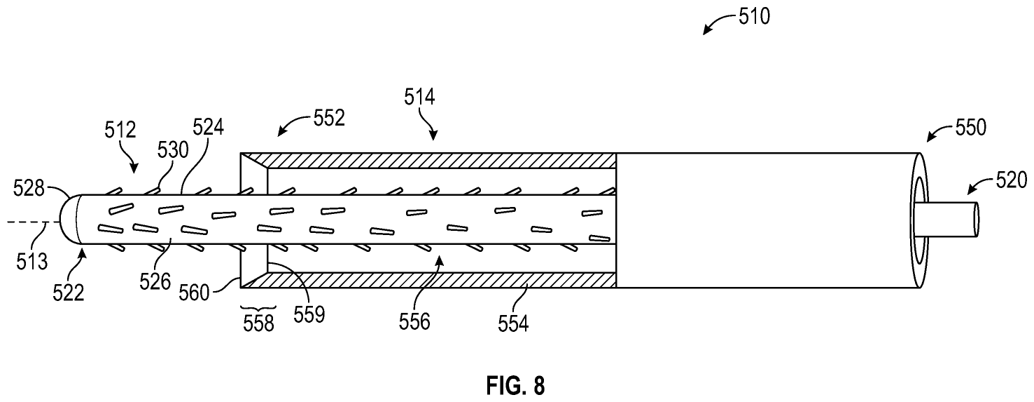

FIG. 8 illustrates a sixth example tissue removal device 510. In this example, the tissue removal device 510 is an elongate member 512 partially disposed within a tubular member 514. The elongate member 512 is similar to the elongate member 12 illustrated in FIG. 1 and described above, except as detailed below. In the illustrated embodiment, the elongate member 512 has a lengthwise axis 513, a proximal end 520, a distal end 522, and a main body 524 that defines a central shaft 526, a blunted distal tip 528, and a plurality of barbs 530. The central shaft 526 extends from the proximal end 520 to the distal end 522. In the illustrated embodiment, each barb of the plurality of barbs 530 extends from the central shaft 526 and away from the lengthwise axis 513 of the elongate member 512. Each barb of the plurality of barbs 530 can optionally be sharpened and electropolished to provide a sharp cutting surface. In an alternative embodiment, an elongate member can include a roughened surface along a portion of the elongate member that extends from the distal end toward the proximal end, or from a location between the distal end and the proximal end toward the proximal end. This alternative embodiment can include the barbs described herein, or omits the barbs described herein.

The tubular member 514 disposed over the elongate member 512 and has a proximal end 550, a distal end 552, and a main body 554 that defines a lumen 556, a tapered wall portion 558, and a sharp distal tip 560. The lumen 556 extends from the proximal end 550 to the distal end 552 and is sized to receive the elongate member 512. The tapered wall portion 558 extends from a location 559 disposed between the proximal end 550 and the distal end 552 to the distal end 552 and tapers from the location 559 to the distal end 552. The sharp distal tip 560 is disposed on the distal end 552. Optionally, an elongate member and/or tubular member can include one or more markers, such as at, or near, the distal end of the elongate member or tubular member, along the length of the elongate member or tubular member, and any other location considered suitable for a particular embodiment.

To remove tissue from a bodily passage, the elongate member 512 and tubular member 514 are positioned adjacent, or within, the tissue and the elongate member 512 is advanced distal to the distal end 552 of the tubular member 514. This can be accomplished by applying a distally-directed force on the elongate member 512 while maintaining the position of the tubular member 514, applying a proximally-directed force on the tubular member 514 while maintaining the position of the elongate member 512, or applying a distally-directed force on the elongate member 512 while applying a proximally-directed force on the tubular member 514. Once the elongate member 512 is disposed distal to the distal end 552 of the tubular member 514, an oscillating or vibrating force is applied to the elongate member 512 while applying a distally-directed force on the elongate member 512, applying a distally-directed force on the elongate member 512 and a distally-directed force on the tubular member 514, applying a distally-directed force on the elongate member 512 while applying a proximally-directed force on the tubular member 514 such that tissue advances into the lumen 556 defined by the tubular member 514 and is mechanically disrupted. Alternatively, the application of an oscillating or vibrating force can be omitted and other steps completed to remove tissue. The sharp distal tip 560 cuts the tissue as it is pulled into the lumen 556, which can then be removed from the bodily passage by aspiration or suction through the lumen 556 or through a lumen defined by a device through which the tubular member 514 and elongate member 512 are disposed. The application of an oscillating or vibrating force on the elongate member 512 can be accomplished using any suitable method or technique. In the embodiment illustrated, an oscillating or vibrating force can be applied using the system 570 illustrated in FIG. 9, as described in more detail herein. Alternatively, an oscillating or vibrating force can be applied manually by a user.

Use of the tissue removal devices described herein provide a mechanism for removing tissue, such as the edges of an abnormal growth being removed from a body (e.g., edges of a tumor) if other techniques (e.g., ablative techniques) have been attempted but were not successful in removing all of the abnormal growth.

Oscillation or vibration forces can be applied to an elongate member using any suitable device or system and selection of a suitable device or system can be based on various considerations, including the structural arrangement of a tissue removal device being oscillated or vibrated. FIG. 9 illustrates an example system 570 for applying an oscillating force or a vibrating force on an elongate member 572 attached to a handle 580. The system is configured to resonate the elongate member 572 at a frequency of 15-25 kHz using any suitable method or technique. For example, an elongate member of a tissue removal device can be driven (e.g., oscillated, vibrated) using piezoelectric crystals (e.g., lead zirconium titanate) on which an electric field is applied. The resulting mechanical forces imparted on the tissue by the elongate member cause shearing and cavitation to ablate the target tissue. Any heating and devitalization of tissue can be monitored by MRI scanning. While 15-25 kHz has been described, other frequencies are considered suitable. Alternatively, oscillation or vibration of an elongate member can be accomplished using a pneumatic or hydraulic actuation of gears (e.g., driven manually by the operator or otherwise) positioned within a handle to which an elongate member is attached, or by using materials that naturally vibrate under MRI. Alternately, a handle can be omitted from a tissue removal device and oscillation or vibration of the elongate member can be accomplished manually.

Various methods of performing interventional medical treatment under MRI are described herein. While the methods described herein are shown and described as a series of acts, it is to be understood and appreciated that the methods are not limited by the order of acts, as some acts may in accordance with these methods may be omitted, occur in the order shown and/or described, occur in different orders, and/or occur concurrently with other acts described herein.

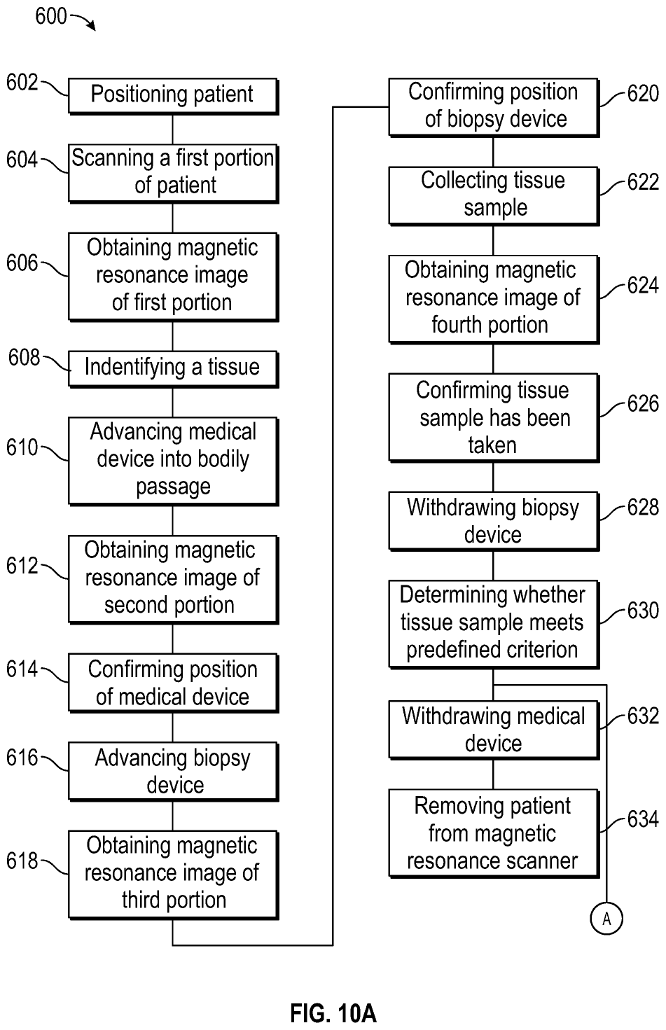

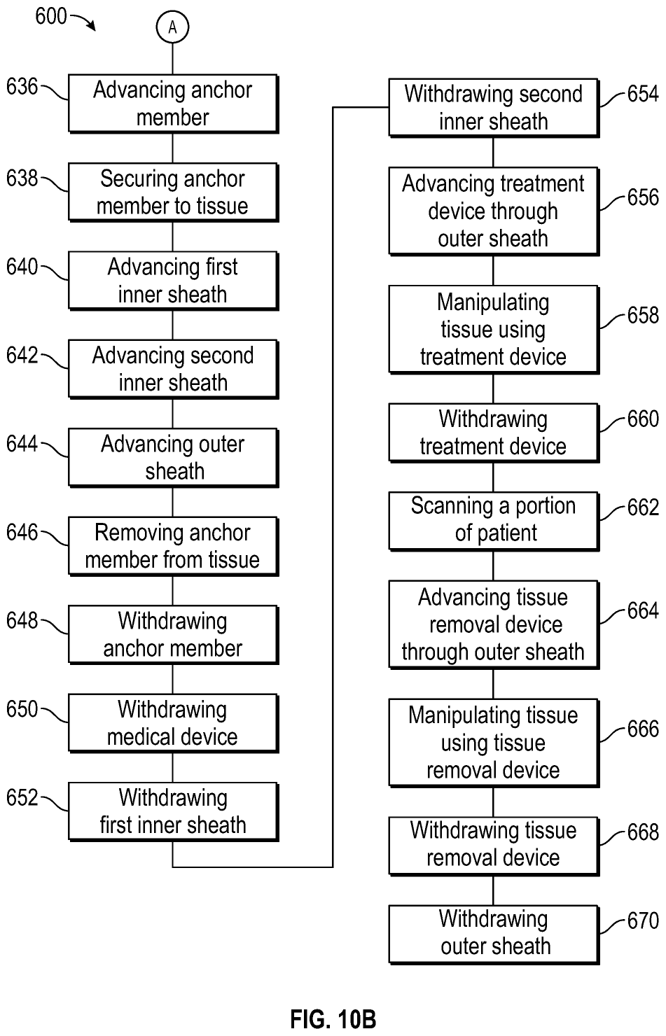

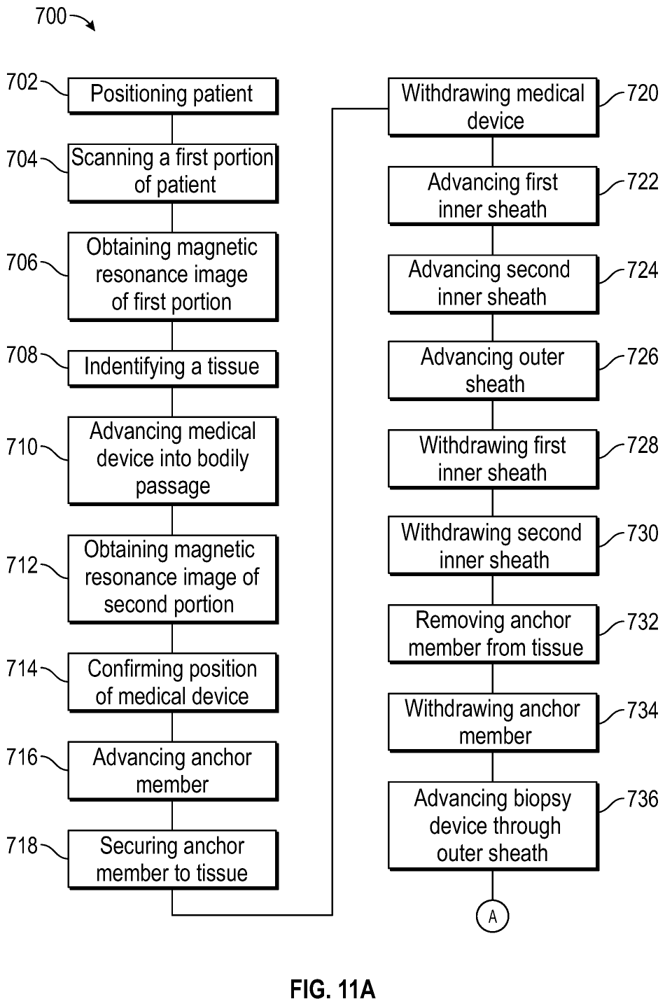

FIGS. 10A and 10B illustrate a schematic illustration of an example method 600 of performing treatment under MRI.

An initial step 602 comprises positioning a patient within a magnetic resonance scanner. Another step 604 comprises scanning a first portion of the patient using the magnetic resonance scanner. Another step 606 comprises obtaining a magnetic resonance image of the first portion of the patient. Another step 608 comprises identifying a tissue that has predefined characteristics using the magnetic resonance image. While the patient remains positioned within the magnetic resonance scanner used to scan a portion of the patient, another step 610 comprises advancing a medical device into a bodily passage and to, within, or adjacent to, the tissue while scanning a second portion of the patient that includes the medical device using the magnetic resonance scanner. Another step 612 comprises obtaining a magnetic resonance image of the second portion of the patient that includes the medical device. Another step 614 comprises confirming the position of the medical device within the bodily passage. Another step 616 comprises advancing a biopsy device through the medical device and to the tissue while scanning a third portion of the patient that includes the biopsy device using the magnetic resonance scanner. Another step 618 comprises obtaining a magnetic resonance image of the third portion of the patient that includes the biopsy device. Another step 620 comprises confirming the position of the biopsy device. Another step 622 comprises collecting a tissue sample from the tissue using the biopsy device while scanning a fourth portion of the patient that includes the biopsy device and the tissue using the magnetic resonance scanner. Another step 624 comprises obtaining a magnetic resonance image of the fourth portion of the patient that includes the biopsy device. Another step 626 comprises confirming the tissue sample has been collected. Another step 628 comprises withdrawing the biopsy device and the tissue sample through the medical device. Another step 630 comprises determining whether the tissue sample meets a predefined criterion. If the tissue sample does not meet the predefined criterion, additional steps comprise 632 withdrawing the medical device from the bodily passage and 634 removing the patient from the magnetic resonance scanner. If the tissue sample meets the predefined criterion, another step 636 comprises advancing an anchor member through the medical device through which the biopsy device was advanced and to the tissue while the patient remains positioned within the magnetic resonance scanner. Another step 638 comprises securing the anchor member to the tissue to retain the position of the medical device relative to the tissue. Another step 640 comprises advancing a first inner sheath over the medical device and toward the tissue to dilate the bodily passage. Another step 642 comprises advancing a second inner sheath over the first inner sheath and toward the tissue to dilate the bodily passage. Another step 644 comprises advancing an outer sheath over the second inner sheath and toward the tissue to dilate the bodily passage. Another step 646 comprises removing the anchor member from the tissue. Another step 648 comprises withdrawing the anchor member from the bodily passage. Another step 650 comprises withdrawing the medical device from the bodily passage. Another step 652 comprises withdrawing the first inner sheath from the bodily passage. Another step 654 comprises withdrawing the second inner sheath from the bodily passage. Another step 656 comprises advancing a treatment device through the outer sheath and to the tissue. Another step 658 comprises manipulating the tissue using the treatment device. Another step 660 comprises withdrawing the treatment device from the outer sheath. Another step 662 comprises scanning a first portion of the patient using the magnetic resonance scanner. Another step 664 comprises advancing a tissue removal device through the outer sheath and to the tissue. Another step 666 comprises manipulating the tissue using the tissue removal device. Another step 668 comprises withdrawing the tissue removal device from the outer sheath. Another step 670 comprises withdrawing the outer sheath from the bodily passage.

Step 602 can be accomplished by positioning a patient within any suitable magnetic resonance scanner, such as conventional magnetic resonance scanners, magnetic resonance scanners that utilize 0.55 T fields, 1.5 T fields, 3 T fields, fields between about 0.055 T and 1.5 T, fields less than 1 T, and any other magnetic resonance scanner considered suitable for a particular embodiment.

Step 604 can be accomplished by scanning any suitable portion of a patient and selection of a suitable portion of a patient to scan can be based on various considerations, including the treatment intended to be performed. Examples of portions of a patient considered suitable to scan include the extremities (e.g., arms, legs), chest, breast, spine, neck, head, abdomen, pelvis, prostate, peri-prostatic structures, tissue surrounding the portions described herein, and/or any other portion of the patient considered suitable for a particular embodiment.

Step 606 can be accomplished by obtaining the magnetic resonance image from the magnetic resonance scanner used in step 602.

Step 608 can be accomplished by reviewing the magnetic resonance image obtained in step 606 and utilizing conventional techniques and/or methods to determine whether tissue has predefined characteristics (e.g., tissue has characteristics indicative of cancer, is a lesion, abnormal mass). Furthermore, the margins (e.g., borders) of any tissue (e.g., abnormal mass, lesions) can be identified and used in further steps, as described herein, to remove and/or treat the tissue.

Step 610 can be accomplished using any suitable medical device, such as the medical devices and/or cannulas described in U.S. Patent Application No. 63/135,801, filed on Jan. 11, 2021, which is hereby incorporated by reference in its entirety for the purpose of describing medical devices considered suitable to complete a step within a method of treatment. Step 610 can be accomplished by applying a distally-directed force on the medical device such that a distal end of the medical device is advanced into a bodily passage and to, within, or adjacent to, the tissue that has been identified as having the predefined characteristics. A bodily passage can include any suitable portion of a body, including existing bodily passages, bodily lumens, and/or bodily passages created through tissues layers and/or fascia using a device described herein. Step 610 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the medical device, portions that include the tissue that has predefined characteristics and the medical device, and any other portion of the patient considered suitable for a particular embodiment. For example, a second portion of the patient can be the same as, or different than, the first portion of the patient.

Step 612 can be accomplished by obtaining the magnetic resonance image from the magnetic resonance scanner used in step 602.

Step 614 can be accomplished by reviewing the magnetic resonance image obtained in step 612 and confirming the medical device is positioned at a desired location within the bodily passage (e.g., at, within, or adjacent to, the tissue that has been identified as having the predefined characteristics). This can be accomplished by visualizing one or more markers included on the medical device, as described herein. If the medical device is not positioned at a desired location, an optional step comprises manipulating the position of the medical device.

Step 616 can be accomplished by applying a distally-directed force on the biopsy device such that a distal end of the biopsy device is advanced into a lumen defined by the medical device, through the lumen defined by the medical device, and to the tissue that has been identified as having the predefined characteristics. Step 616 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the biopsy device, portions that include the tissue that has predefined characteristics and the biopsy device, and any other portion of the patient considered suitable for a particular embodiment. For example, a third portion of the patient can be the same as, or different than, the first portion of the patient and/or the second portion of the patient.

Step 616, step 622, and step 628 can be accomplished using any suitable biopsy device, such as MRI compatible biopsy devices, the Echotip ProCore provided by Cook Medical, the Echotip Ultra provided by Cook Medical, and any other biopsy device considered suitable for a particular embodiment. Alternatively, step 616, step 622, and step 628 can be completed using a biopsy device that is not MRI compatible. This alternative step can comprise advancing the biopsy device through the medical device and to the tissue without scanning a portion of the patient that includes the biopsy device using the magnetic resonance scanner. An alternative to step 628 can comprise collecting a tissue sample using the biopsy device without scanning a portion of the patient that includes the biopsy device and the tissue using the magnetic resonance scanner.

Step 618 can be accomplished by obtaining the magnetic resonance image from the magnetic resonance scanner used in step 602.

Step 620 can be accomplished by reviewing the magnetic resonance image obtained in step 618 and confirming the biopsy device is positioned at a desired location within the bodily passage (.e.g., at, within, or adjacent to, the tissue that has been identified as having the predefined characteristics). If the biopsy device is not positioned at a desired location, an optional step comprises manipulating the position of the biopsy device.

Step 622 can be accomplished using the biopsy device and conventional methods of obtaining a tissue sample using a biopsy device. Step 622 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the biopsy device, portions that include the tissue that has predefined characteristics and the biopsy device, and any other portion of the patient considered suitable for a particular embodiment. For example, a fourth portion of the patient can be the same as, or different than, the first portion of the patient, the second portion of the patient, and/or the third portion of the patient.

Step 610, step 616, and/or step 622 can optionally be conducted in combination with performing an ultrasound on the portion of the patient that includes the medical device and/or biopsy device. In embodiments in which an ultrasound image is obtained, the magnetic resonance image obtained can be electronically fused with a real-time ultrasound image (e.g., transrectal ultrasound image of a prostate).

Step 624 can be accomplished by obtaining the magnetic resonance image from the magnetic resonance scanner used in step 602.

Step 626 can be accomplished by reviewing the magnetic resonance image obtained in step 624 and confirming the biopsy device has collected the tissue sample (e.g., the tissue that has been identified as having the predefined characteristics). If the biopsy device has not collected the tissue sample, optional steps comprise repeating step 616, step 618, step 620, step 622, step 624, and/or step 626.

Step 606, step 612, step 618, and/or step 624 can comprise obtaining a single still image. Alternatively, step 604, step 610, step 616, and/or step 622 can be repeated any desired number of times such that step 606, step 612, step 618, and/or step 624 comprises obtaining multiple magnetic resonance images of a portion that can be grouped as a cine to show motion.

Step 628 can be accomplished by applying a proximally-directed force on the biopsy device such that it is withdrawn from the lumen defined by the medical device. In an alternative embodiment, step 616, step 618, step 620, step 622, step 624, step 626, and step 628 can be omitted from method 600, and other methods described herein, and the medical device advanced in step 610 can be utilized to obtain a biopsy and steps similar to those described with respect to a biopsy device can be completed utilizing the medical device. In another alternative embodiment, an anchor can be placed, as described herein, the medical device withdrawn, and the anchor used to track a biopsy device to the tissue.

Step 630 can be accomplished using any technique or method considered suitable to determine whether tissue meets predefined criterion. For example, step 630 can utilize conventional techniques and methods for determining whether a tissue sample is malignant, such as frozen section and/or other cytological methods.

Each of step 606, step 608, step 610, step 612, step 614, step 616, step 618, step 620, step 622, step 624, step 626, step 628, and/or step 630 can be accomplished without removing the patient from the magnetic resonance scanner within which the patient is positioned in step 602.

Step 632 can be accomplished by applying a proximally-directed force on the medical device until it is withdrawn from the bodily passage. Step 634 can be accomplished by withdrawing the patient from the magnetic resonance scanner such that the patient is free of the magnetic resonance scanner.

Step 636 can be accomplished using any suitable anchor member, such as the anchor members described in U.S. Patent Application No. 63/135,801, filed on Jan. 11, 2021, which is hereby incorporated by reference in its entirety for the purpose of describing anchors considered suitable to complete a step in a method of treatment. Step 636 can be accomplished by applying a distally-directed force on the anchor member such that a distal end of the anchor member is advanced into a lumen defined by the medical device, through the lumen defined by the medical device, and to the tissue, within, or adjacent to the tissue. Optionally, step 636 can be completed while scanning a fifth portion of the patient that includes the anchor member using the magnetic resonance scanner and additional steps that can be completed subsequent to this optional step include obtaining a magnetic resonance image of the fifth portion of the patient that includes the anchor member, and confirming the position of the anchor member. In embodiments in which it is desired to utilize the magnetic resonance scanner on a second patient that is different from the patient, optional steps that can be completed subsequent to step 638 include removing the patient from the magnetic resonance scanner while maintaining the position of the patient on a surface, positioning a second patient within the magnetic resonance scanner, scanning a portion of the second patient using the magnetic resonance scanner, obtaining a magnetic resonance image of the portion of the second patient, removing the second patient from the magnetic resonance scanner, and repositioning the patient within the magnetic resonance scanner.

Completion of step 638 of securing the anchor member into the tissue will depend on the structural configuration of the anchor member. Examples of suitable actions that can be performed for this step include, but are not limited to, axially advancing the anchoring member through a lumen defined by a medical device until a portion of the anchor member (e.g., barb) becomes disposed within the tissue, or applying torque on the anchoring member until a portion of the anchor member becomes disposed within the tissue. Alternatively, step 638 can comprise anchoring the anchor member into a second, different tissue disposed adjacent to or within the tissue. Step 638 allows for the medical device to be utilized as a guide rail to a target site such that one or more other devices can be advanced over the medical device to a treatment site. Optionally, step 638 can be completed while scanning a sixth portion of the patient that includes the anchor member using the magnetic resonance scanner and additional steps that can be completed subsequent to this optional step include obtaining a magnetic resonance image of the sixth portion of the patient that includes the anchor member, and confirming the position of the anchor member. Step 638 allows for site retention such that the medical device and/or anchor member stays positioned relative to the tissue after biopsy and prior to treatment and can be used to direct a treatment device to the tissue, as described in more detail herein.

Step 640 can be accomplished by applying a distally-directed force on the first inner sheath such that a distal end of the first inner sheath is advanced over the medical device and to the tissue. Optionally, step 640 can be completed while scanning a seventh portion of the patient that includes the first inner sheath using the magnetic resonance scanner and additional steps that can be completed subsequent to this optional step include obtaining a magnetic resonance image of the seventh portion of the patient that includes the first inner sheath, and confirming the position of the first inner sheath.

Step 642 can be accomplished by applying a distally-directed force on the second inner sheath such that a distal end of the second inner sheath is advanced over the first inner sheath and to the tissue. Optionally, step 642 can be completed while scanning an eighth portion of the patient that includes the second inner sheath using the magnetic resonance scanner and additional steps that can be completed subsequent to this optional step include obtaining a magnetic resonance image of the eighth portion of the patient that includes the second inner sheath, and confirming the position of the second inner sheath.

Step 644 can be accomplished by applying a distally-directed force on the outer sheath such that a distal end of the outer sheath is advanced over the second inner sheath and to the tissue. Optionally, step 644 can be completed while scanning a ninth portion of the patient that includes the outer sheath using the magnetic resonance scanner and additional steps that can be completed subsequent to this optional step include obtaining a magnetic resonance image of the ninth portion of the patient that includes the outer sheath, and confirming the position of the outer sheath.

Completion of step 640, step 642, and 644 results in sequential dilation of the bodily passage under MRI. While only first and second inner sheaths and a single outer sheath has been described as accomplishing sequential dilation, any suitable number of inner sheaths and/or outer sheaths can be used to complete sequential dilation of a bodily passage. Examples of numbers of inner sheaths and/or outer sheaths considered suitable to accomplish sequential dilation includes one, two, a plurality, three, four, and any other number considered suitable for a particular embodiment. In embodiment in which sequential dilation is not desired, step 640 can alternatively comprise advancing a dilator over the medical device and toward the tissue to dilate the bodily passage and step 642 and step 644 can be omitted from method 600. In this alternative step, step 640 can be accomplished using any suitable dilator, such as conventional dilators, and/or the dilators described herein.

Completion of step 646 of removing the anchor member from the tissue will depend on the structural configuration of the anchor member. Examples of suitable actions that can be performed for this step include, but are not limited to, applying a proximally-directed force on the anchor member such that it is withdrawn through the lumen defined by a medical device until a portion of the anchor member (e.g., barb) becomes free of the tissue, applying torque to the anchoring member until the portion of the anchor member disposed within the tissue becomes free of the tissue, or applying a distally-directed force on the anchor member such that it is advanced within the lumen defined by a medical device until a portion of the anchor member (e.g., barb) becomes free of the tissue. Alternatively, step 646 can comprise removing the anchor member from a tissue disposed adjacent to or within the tissue. Optionally, step 646 can be completed while scanning a tenth portion of the patient that includes the anchor member using the magnetic resonance scanner and additional steps that can be completed subsequent to this optional step include obtaining a magnetic resonance image of the tenth portion of the patient that includes the anchor member, and confirming the position of the anchor member.

Step 648 can be accomplished by applying a proximally-directed force on the anchor member such that it is withdrawn from the lumen defined by the medical device. In an alternative embodiment, step 636, step 638, step 646, and step 648 can be omitted from method 600 and similar steps can be omitted from other methods described herein.

Step 650 can be accomplished by applying a proximally-directed force on the medical device until it is withdrawn from the bodily passage.

In an alternative embodiment, step 640 can comprise withdrawing the medical device from the bodily passage, step 642 can comprise advancing a dilator over the anchor member and toward the tissue to dilate the bodily passage, step 644 can comprise removing the anchor member from the tissue, and step 646 can comprise withdrawing the anchor member from the bodily passage. In this alternative embodiment, dilation is accomplished over the anchor member. Sequential dilation can also be accomplished over the anchor member and be completed as described herein utilizing original step 640, step 642, and step 644.

Step 652 can be accomplished by applying a proximally-directed force on the first inner sheath until it is withdrawn from the bodily passage.

Step 654 can be accomplished by applying a proximally-directed force on the second inner sheath until it is withdrawn from the bodily passage.

Step 656 can be accomplished by applying a distally-directed force on the treatment device such that a distal end of the treatment device is advanced into a lumen defined by the outer sheath, or dilator, through the lumen defined by the outer sheath, or dilator, and to the tissue. Any suitable treatment device can be utilized in method 600 and selection of a suitable treatment device can be based on various considerations, such as the intended use of the treatment device. Examples of treatment devices considered suitable to treat tissue for which a tissue sample meets a predefined criterion include dissection tools, optical fibers, optical fibers formed of a material selected from the group consisting of argon, dye, erbium, excimer, Nd:YAG, and CO², optical fibers that include control cables (e.g., ultra-high molecular weight polyethylene, Dyneema) to direct the fibers toward tissue intended to be treated, needles, cannulas, such as those described herein or incorporated by reference, and any other treatment device considered suitable for a particular embodiment. Optionally, step 656 can be completed while scanning an eleventh portion of the patient that includes the treatment device using the magnetic resonance scanner and additional steps that can be completed subsequent to this optional step include obtaining a magnetic resonance image of the eleventh portion of the patient that includes the treatment device, and confirming the position of the treatment device.

Step 658 can be accomplished by physically manipulating the tissue using the treatment device, which can include removing of all or a portion of the tissue, introducing a material into the tissue, applying a treatment to the tissue, and performing any other suitable treatment on the tissue. Examples of treatments considered suitable include laser direct therapy, photodynamic therapy (PDT), chemotherapy, a focal treatment, a radical prostatectomy, infusion of ablative agents, such as acetic acid, ethanol, sclerosants (e.g., sodium tetradecyl sulfate), chemotherapeutic agents, and any other treatment considered suitable for a particular embodiment. Optionally, step 658 can be completed while scanning a twelfth portion of the patient that includes the treatment device using the magnetic resonance scanner and additional steps that can be completed subsequent to this optional step include obtaining a magnetic resonance image of the twelfth portion of the patient that includes the treatment device, and confirming the position of the treatment device. For example, a magnetic resonance scanner can be utilized to determine areas of tissue that become devitalized during ablative therapies. The heating and devitalization of tissue can be monitored such that it occurs in real time and can be used to guide the targeted treatment of the tissue (e.g., prostate).

Step 660 can be accomplished by applying a proximally-directed force on the treatment device until it is withdrawn from the lumen defined by the outer sheath, or dilator.

Step 662 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, the margins of the tissue that has predefined characteristics, and any other portion of the patient considered suitable for a particular embodiment.

Step 664 can be accomplished using any suitable tissue removal device, such as the tissue removal devices described herein, or incorporated by reference. Step 664 can be accomplished by applying a distally-directed force on a tissue removal device such that a distal end of the tissue removal device is advanced into a lumen defined by the outer sheath, or dilator, through the lumen defined by the outer sheath, or dilator, and to the tissue, or margins of the tissue. Optionally, step 664 can be completed while scanning a portion of the patient that includes the tissue removal device using the magnetic resonance scanner and additional steps that can be completed subsequent to this optional step include obtaining a magnetic resonance image of the portion of the patient that includes the tissue removal device, and confirming the position of the tissue removal device.

Step 666 can be accomplished by physically manipulating the tissue using the tissue removal device, which can include removing of all or a portion of the tissue, introducing a material into the tissue, applying a treatment to the tissue, and performing any other suitable treatment on the tissue. For example, step 666 can be accomplished by applying torque to an elongate member of a tissue removal device, as described herein, applying an oscillating or vibrating force on an elongate member of a tissue removal device, as described herein, applying a suction force to lumen defined by an elongate member of a tissue removal device or a lumen through which the tissue removal device is disposed, introducing a flushing fluid or cooling fluid through the lumen defined by an elongate member of a tissue removal device or a lumen through which the tissue removal device is disposed, and/or utilizing a tissue removal device, as described herein. Optionally, step 666 can be completed while scanning a portion of the patient that includes the tissue removal device using the magnetic resonance scanner and additional steps that can be completed subsequent to this optional step include obtaining a magnetic resonance image of the portion of the patient that includes the tissue removal device, and confirming the position of the tissue removal device.

Optionally, step 666 can be repeated. For example, as the space within the body enlarges as step 666 is being completed, more of the tissue removal device (e.g., elongate member) can be introduced into the space created in initial step 666 to increase the area of disruption. Once the space has been maximally enlarged, the tissue removal device and outer sheath are removed and can then be inserted into different locations of the tissue until the entire tissue that has predefined characteristics is removed.

Step 668 can be accomplished by applying a proximally-directed force on the tissue removal device until it is withdrawn from the lumen defined by the outer sheath, or dilator.

In an alternative embodiment, step 656, step 658, and step 660 can be omitted and step 662, step 664, step 666, and step 668 can be utilized to treat the tissue.

Step 670 can be accomplished by applying a proximally-directed force on the outer sheath, or dilator, until it is withdrawn from the bodily passage.

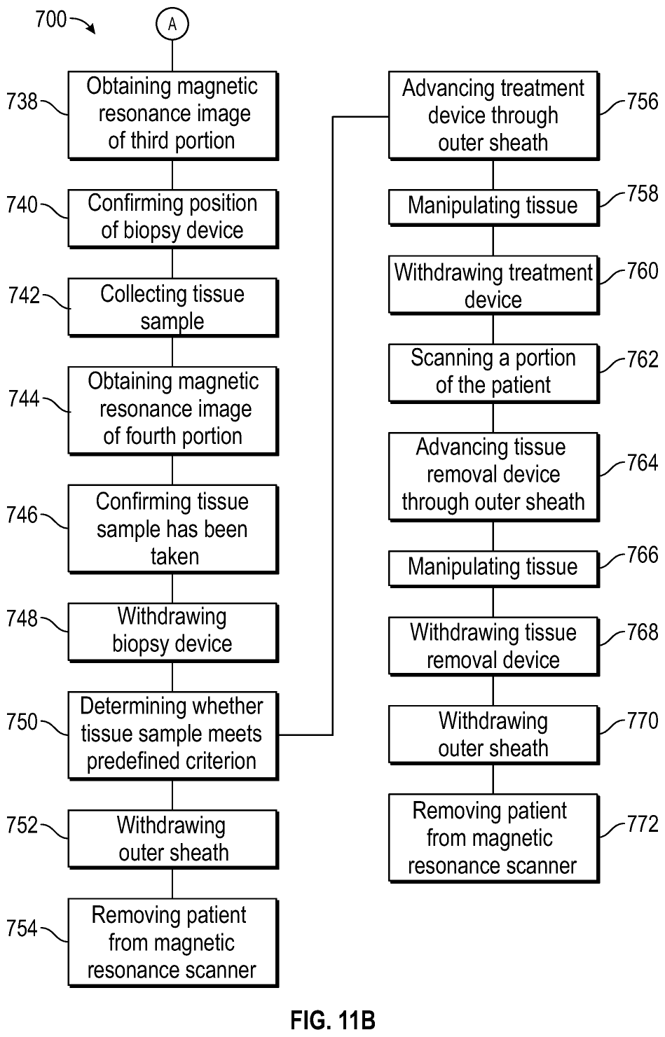

FIGS. 11A and 11B illustrate another schematic illustration of an example method 700 of performing treatment under MRI.

An initial step 702 comprises positioning a patient within a magnetic resonance scanner. Another step 704 comprises scanning a first portion of the patient using the magnetic resonance scanner. Another step 706 comprises obtaining a magnetic resonance image of the first portion of the patient. Another step 708 comprises identifying a tissue that has predefined characteristics using the magnetic resonance image. While the patient remains positioned within the magnetic resonance scanner used to scan a portion of the patient, another step 710 comprises advancing a medical device into a bodily passage and to, within, or adjacent to, the tissue while scanning a second portion of the patient that includes the medical device using the magnetic resonance scanner. Another step 712 comprises obtaining a magnetic resonance image of the second portion of the patient that includes the medical device. Another step 714 comprises confirming the position of the medical device within the bodily passage. Another step 716 comprises advancing an anchor member through the medical device and to the tissue while the patient remains positioned within the magnetic resonance scanner. Another step 718 comprises securing the anchor member to the tissue to retain the position of the anchor member relative to the tissue. Another step 720 comprises withdrawing the medical device from the bodily passage. Another step 722 comprises advancing a first inner sheath over the anchor member and toward the tissue to dilate the bodily passage. Another step 724 comprises advancing a second inner sheath over the first inner sheath and toward the tissue to dilate the bodily passage. Another step 726 comprises advancing an outer sheath over the second inner sheath and toward the tissue to dilate the bodily passage. Another step 728 comprises withdrawing the first inner sheath from the bodily passage. Another step 730 comprises withdrawing the second inner sheath from the bodily passage. Another step 732 comprises removing the anchor member from the tissue. Another step 734 comprises withdrawing the anchor member from the bodily passage. Another step 736 comprises advancing a biopsy device through the outer sheath and to the tissue while scanning a third portion of the patient that includes the biopsy device using the magnetic resonance scanner. Another step 738 comprises obtaining a magnetic resonance image of the third portion of the patient that includes the biopsy device. Another step 740 comprises confirming the position of the biopsy device. Another step 742 comprises collecting a tissue sample from the tissue using the biopsy device while scanning a fourth portion of the patient that includes the biopsy device and the tissue using the magnetic resonance scanner. Another step 744 comprises obtaining a magnetic resonance image of the fourth portion of the patient that includes the biopsy device. Another step 746 comprises confirming the tissue sample has been collected. Another step 748 comprises withdrawing the biopsy device and the tissue sample through the outer sheath. Another step 750 comprises determining whether the tissue sample meets a predefined criterion. If the tissue sample does not meet the predefined criterion, additional steps comprise 752 withdrawing the outer sheath from the bodily passage and 754 removing the patient from the magnetic resonance scanner. If the tissue sample meets the predefined criterion, another step 756 comprises advancing a treatment device through the outer sheath and to the tissue. Another step 758 comprises manipulating the tissue using the treatment device. Another step 760 comprises withdrawing the treatment device from the outer sheath. Another step 762 comprises scanning a first portion of the patient using the magnetic resonance scanner. Another step 764 comprises advancing a tissue removal device through the outer sheath and to the tissue. Another step 766 comprises manipulating the tissue using the tissue removal device. Another step 768 comprises withdrawing the tissue removal device from the outer sheath. Another step 770 comprises withdrawing the outer sheath from the bodily passage. Another step 772 comprises removing the patient from the magnetic resonance scanner.

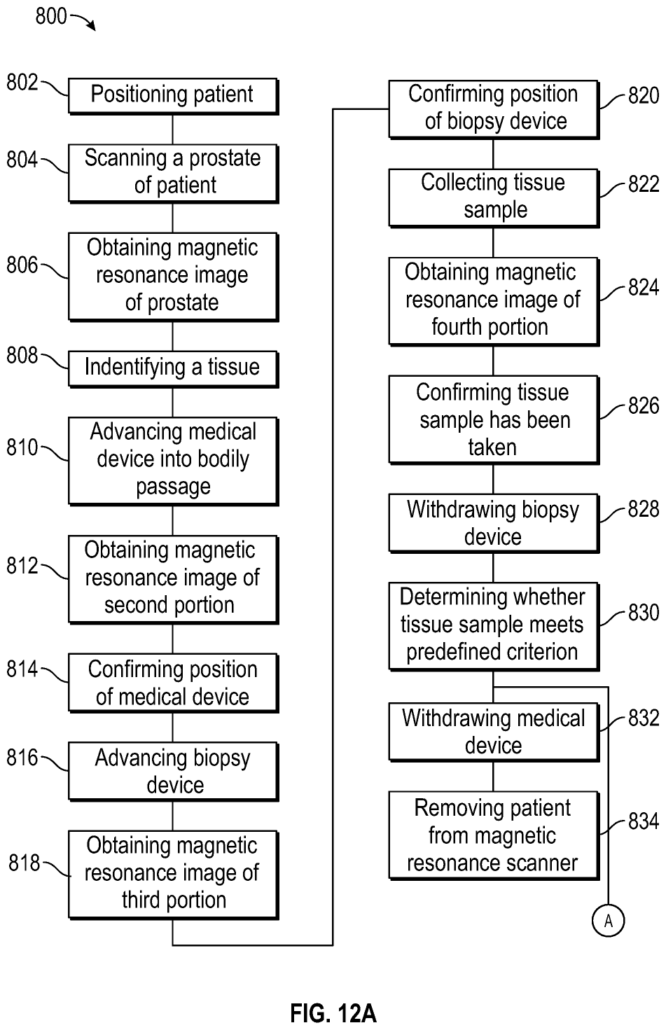

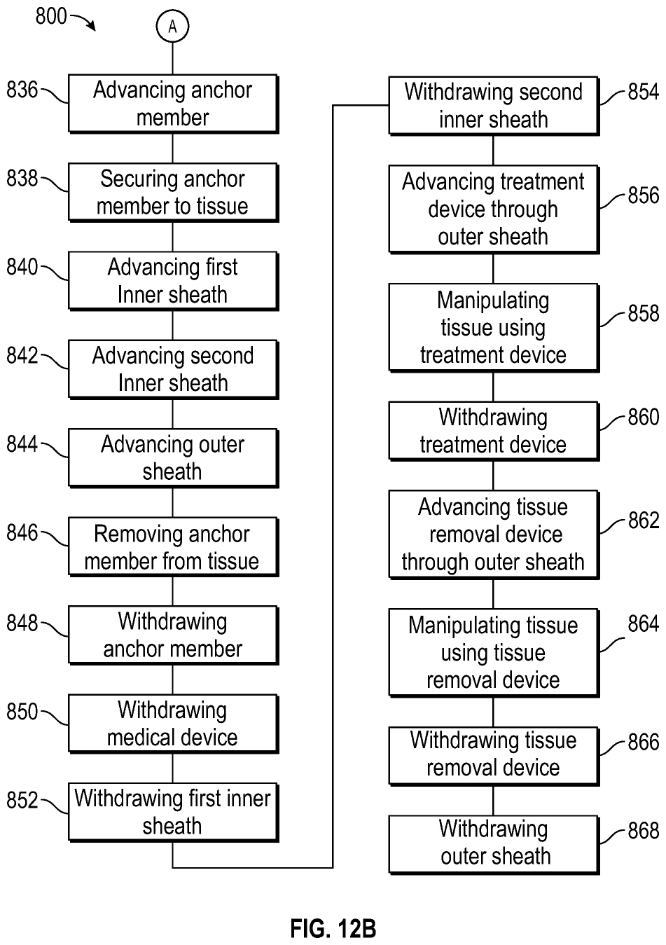

FIGS. 12A and 12B illustrate another schematic illustration of an example method 800 of performing treatment on a prostate under MRI.

An initial step 802 comprises positioning a patient within a magnetic resonance scanner. Another step 804 comprises scanning a prostate and surrounding tissue of the patient using the magnetic resonance scanner. Another step 806 comprises obtaining a magnetic resonance image of the prostate and surrounding tissue of the patient. Another step 808 comprises identifying a tissue within the magnetic resonance image that has predefined characteristics. While the patient remains positioned within the magnetic resonance scanner used to scan the prostate and surrounding tissue, another step 810 comprises advancing a medical device into a bodily passage and to the tissue while scanning a first portion of the patient that includes the medical device using the magnetic resonance scanner. Another step 812 comprises obtaining a magnetic resonance image of the second portion of the patient that includes the medical device. Another step 814 comprises confirming the position of the medical device within the bodily passage. Another step 816 comprises advancing a biopsy device through the medical device and to the tissue while scanning a third portion of the patient that includes the biopsy device using the magnetic resonance scanner. Another step 818 comprises obtaining a magnetic resonance image of the third portion of the patient that includes the biopsy device. Another step 820 comprises confirming the position of the biopsy device. Another step 822 comprises collecting a tissue sample from the tissue using the biopsy device while scanning a fourth portion of the patient that includes the biopsy device and the tissue using the magnetic resonance scanner. Another step 824 comprises obtaining a magnetic resonance image of the fourth portion of the patient that includes the biopsy device. Another step 826 comprises confirming the tissue sample has been collected. Another step 828 comprises withdrawing the biopsy device and the tissue sample through the medical device. Another step 830 comprises determining whether the tissue sample meets a predefined criterion. If the tissue sample does not meet the predefined criterion, additional steps comprise 832 withdrawing the medical device from the bodily passage and 834 removing the patient from the magnetic resonance scanner. If the tissue sample meets the predefined criterion, another step 836 comprises advancing an anchor member through the medical device through which the biopsy device was advanced and to the tissue while the patient remains positioned within the magnetic resonance scanner. Another step 838 comprises securing the anchor member to the tissue to retain the position of the medical device relative to the tissue. Another step 840 comprises advancing a first inner sheath over the medical device and toward the tissue to dilate the bodily passage. Another step 842 comprises advancing a second inner sheath over the first inner sheath and toward the tissue to dilate the bodily passage. Another step 844 comprises advancing an outer sheath over the second inner sheath and toward the tissue to dilate the bodily passage. Another step 846 comprises removing the anchor member from the tissue. Another step 848 comprises withdrawing the anchor member from the bodily passage. Another step 850 comprises withdrawing the medical device from the bodily passage. Another step 852 comprises withdrawing the first inner sheath from the bodily passage. Another step 854 comprises withdrawing the second inner sheath from the bodily passage. Another step 856 comprises advancing a treatment device through the outer sheath and to the tissue. Another step 858 comprises manipulating the tissue using the treatment device. Another step 860 comprises withdrawing the treatment device from the outer sheath. Another step 862 comprises advancing a tissue removal device through the outer sheath and to the tissue. Another step 864 comprises manipulating the tissue using the tissue removal device. Another step 866 comprises withdrawing the tissue removal device from the outer sheath. Another step 868 comprises withdrawing the outer sheath from the bodily passage.

Method 600, method 700, and method 800 are considered advantageous at least because each step of method 600, method 700, and method 800 can be performed during a single patient visit and using the same magnetic resonance scanner, which increases efficiency and reduces the number of patient visits and procedures performed. This results in a set of procedures in which a physician can visualize, diagnose, and treat a patient in a single patient visit. Furthermore, these methods are considered advantageous at least because they provide methods for removing margins of abnormal tissue (e.g., tumor) if other treatment techniques have been unsuccessful or cannot properly remove the margins. For example, MR offers high spatial and temporal resolution in real time that can image tissue (e.g., prostate) and assess it both from a functional aspect as well as morphologically. The methods described herein provide for non-invasive abnormal tissue (e.g., tumor) detection, staging, and consequent direction of biopsy and interventional therapies. They can be used to guide physicians to the desired treatment strategies for an individual patient since the imaging quality of MRI is superior as compared to ultrasound. The higher resolution procedures described herein lead to a better outcome for the patient, faster recovery time, less perioperative pain, and less blood loss as compared to open surgical techniques allowing a patient to return to normal activity in a shorter period of time. While some steps have been described as being completed while scanning a portion of the patient using a magnetic resonance scanner and other steps have not been described as being performed while scanning a portion of the patient using a magnetic resonance scanner, any step described herein can be completed while scanning a portion of a patient using the magnetic resonance scanner, and/or an ultrasound device or without scanning a portion of a patient using a magnetic resonance scanner. In embodiments in which an ultrasound image is obtained, the magnetic resonance image obtained can be electronically fused with a real-time ultrasound image (e.g., transrectal ultrasound image of a prostate). While some steps have been described as being completed while scanning a portion of the patient using a magnetic resonance scanner, this step can be broken into two separate steps such that a subsequent step of scanning a portion of a patient using the magnetic resonance scanner can be accomplished. Furthermore, any step which is completed while scanning a portion of the patient using the magnetic resonance scanner can comprise obtaining a single still image and be repeated any desired number of times to obtain multiple magnetic resonance images that can be grouped as a cine to show motion and/or any step which is completed while scanning a portion of the patient using the magnetic resonance scanner can comprise obtaining a live image, such as being completed under live real-time MRI visualization.

While a number of methods have been described herein, it will be appreciated that the method may be a non-invasive method that does not require an invasive intervention by a medical professional. For example, a method may be carried out within a body lumen or passageway, such as the ear canal or a nasal passage, for example in order to place a device within such a passageway. Equally, methods may be implemented on a cadaver or artificial body parts for example for training purposes. Moreover, the skilled person will appreciate that the methods described herein may not be used on the human or animal body at all, but may be used in order to view other types of devices using MRI imaging techniques, for example in an industrial setting.

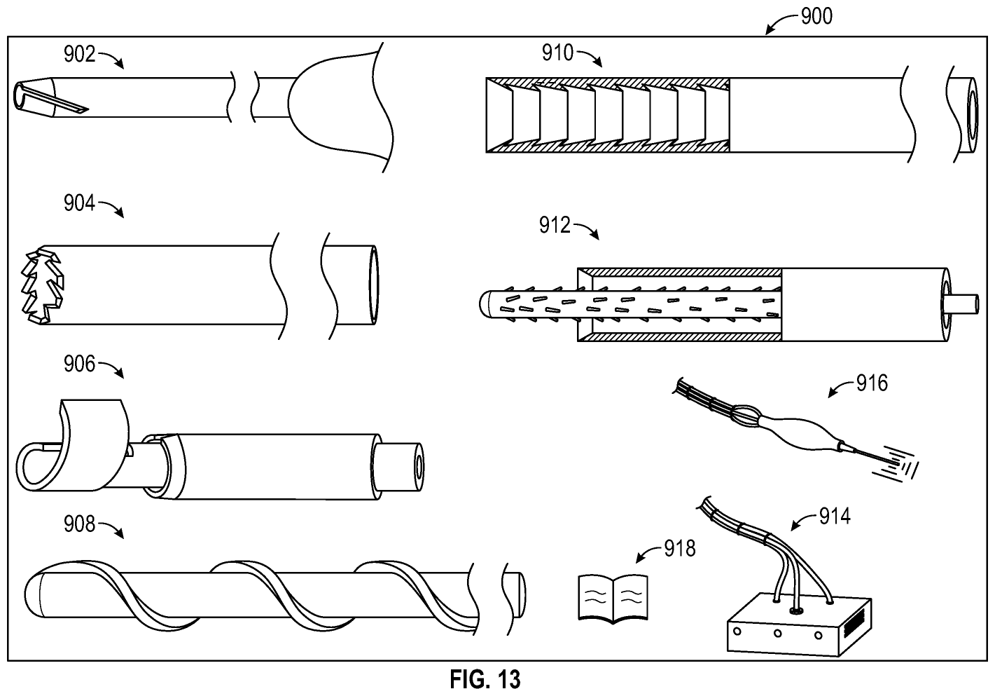

FIG. 13 illustrates an exemplary kit 900 comprising a first tissue removal device 902 according to an embodiment, such as tissue removal device 10 illustrated in FIG. 1; a tissue removal device 904 according to an embodiment, such as tissue removal device 110 illustrated in FIG. 2; a third tissue removal device 906 according to an embodiment, such as tissue removal device 210 illustrated in FIGS. 3, 4, and 5; a fourth tissue removal device 908 according to an embodiment, such as tissue removal device 310 illustrated in FIG. 6; a fifth tissue removal device 910 according to an embodiment, such as tissue removal device 410 illustrated in FIG. 7; a sixth tissue removal device 912 according to an embodiment, such as tissue removal device 510 illustrated in FIG. 8; a system for use with a tissue removal device 914 according to an embodiment, such as system 570 illustrated in FIG. 9; a seventh tissue removal device 916 according to an embodiment, such as tissue removal device 572, 580 illustrated in FIG. 9; and instructions for use 918.

While kit 900 has been illustrated as including seven tissue removal devices 902, 904, 906, 908, 910, 912, 914, and a system for use with a tissue removal device, any suitable number, and type, of tissue removal devices and/or systems for use with a tissue removal device can be included in a kit. Selection of a suitable number of tissue removal devices and/or systems for use with a tissue removal device to include in a kit according to a particular embodiment can be based on various considerations, such as the treatment intended to be performed. Examples of numbers of tissue removal devices and/or systems for use with a tissue removal device considered suitable to include in a kit include at least one, one, two, a plurality, three, four, five, six, seven, eight, nine, ten, more than ten, and any other number considered suitable for a particular embodiment.

Furthermore, while tissue removal device 10, tissue removal device 110, tissue removal device 210, tissue removal device 310, tissue removal device 410, tissue removal device 510, system for use with a tissue removal device 570, and tissue removal device 572, 580 have been illustrated as included in kit 900, any suitable tissue removal device and/or system can be included in a kit. Selection of a suitable tissue removal device and/or system to include in a kit according to a particular embodiment can be based on various considerations, such as the treatment intended to be performed. Examples of tissue removal devices and systems considered suitable to include in a kit include tissue removal device 10, tissue removal device 110, tissue removal device 210, tissue removal device 310, tissue removal device 410, tissue removal device 510, system 570, tissue removal device 572, 580, and/or any other tissue removal device and/or system considered suitable for a particular embodiment.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangement of elements and steps disclosed herein have been selected by the inventor(s) simply to describe and illustrate examples of the invention and are not intended to limit the scope of the invention or its protection, which is to be given the full breadth of the appended claims and any and all equivalents thereof.

What is claimed is:

1. A method of performing treatment under magnetic resonance imaging comprising:
positioning a patient within a magnetic resonance scanner;
scanning a first portion of the patient using the magnetic resonance scanner;
obtaining a magnetic resonance image of the first portion of the patient;
identifying a tissue that has predefined characteristics using the magnetic resonance image;
while the patient is positioned within the magnetic resonance scanner used to scan the first portion of the patient, advancing a medical device into a bodily passage and to the tissue while scanning a second portion of the patient that includes the medical device using the magnetic resonance scanner;
obtaining a magnetic resonance image of the second portion of the patient that includes the medical device;
confirming the position of the medical device within the bodily passage;
advancing a biopsy device through the medical device and to the tissue while scanning a third portion of the patient that includes the biopsy device using the magnetic resonance scanner;
obtaining a magnetic resonance image of the third portion of the patient that includes the biopsy device;
confirming the position of the biopsy device within the bodily passage;
collecting a tissue sample from the tissue using the biopsy device while scanning a fourth portion of the patient that includes the biopsy device and the tissue using the magnetic resonance scanner;
obtaining a magnetic resonance image of the fourth portion of the patient that includes the biopsy device;
confirming the tissue sample has been collected;
withdrawing the biopsy device and the tissue sample through the medical device;
determining whether the tissue sample meets a predefined criterion;
if the tissue sample meets the predefined criterion, advancing an anchor member through the medical device through which the biopsy device was advanced and to the tissue while the patient remains positioned within the magnetic resonance scanner;
securing the anchor member to the tissue to retain the position of the medical device relative to the tissue;
advancing a first inner sheath over the medical device and toward the tissue to dilate the bodily passage;
advancing a second inner sheath over the first inner sheath and toward the tissue to dilate the bodily passage;
advancing an outer sheath over the second inner sheath and toward the tissue to dilate the bodily passage;
removing the anchor member from the tissue;
withdrawing the anchor member from the bodily passage;
withdrawing the medical device from the bodily passage;
withdrawing the first inner sheath from the bodily passage;
withdrawing the second inner sheath from the bodily passage;
advancing a treatment device through the outer sheath and to the tissue;
manipulating the tissue using the treatment device;
withdrawing the treatment device from the outer sheath;
advancing a tissue removal device through the outer sheath and to the tissue;
manipulating the tissue using the tissue removal device;
withdrawing the tissue removal device from the outer sheath; and
withdrawing the outer sheath from the bodily passage.

2. The method of claim 1, wherein advancing a tissue removal device through the outer sheath and to the tissue comprises advancing a tissue removal device through the outer sheath while scanning a fifth portion of the patient that includes the tissue removal device using the magnetic resonance scanner.

3. The method of claim 2, wherein advancing a tissue removal device is conducted in combination with performing an ultrasound on the fifth portion of the patient that includes the tissue removal device.

4. The method of claim 1, wherein determining whether the tissue sample meets a predefined criterion comprises determining whether the tissue sample is malignant.

5. The method of claim 1, wherein manipulating the tissue using the tissue removal device comprises applying torque to a portion of the tissue removal device.

6. The method of claim 1, wherein manipulating the tissue using the tissue removal device comprises applying an oscillating force on a portion of the tissue removal device.

7. The method of claim 1, wherein manipulating the tissue using the tissue removal device comprises applying a suction force to a portion of the tissue removal device.

8. The method of claim 1, wherein manipulating the tissue using the tissue removal device comprises introducing a flushing fluid through a portion of the tissue removal device.

9. The method of claim 1, wherein the tissue removal device comprises an elongate member having a distal end and a sharp edge disposed on the distal end.

10. The method of claim 9, wherein the tissue removal device comprises a handle attached to the elongate member, the handle adapted to move the elongate member relative to the tissue.

11. The method of claim 1, wherein the tissue removal device includes a plurality of markers.

12. The method of claim 11, wherein each marker of the plurality of markers is formed of a ferromagnetic material.

13. The method of claim 1, wherein the tissue removal device comprises an elongate member having a proximal end, a distal end, and a main body defining a slot extending from the distal end toward the proximal end.

14. The method of claim 1, wherein the tissue removal device comprises an elongate member having a distal end with a plurality of sharpened edges.

15. The method of claim 1, wherein the tissue removal device comprises an elongate member having a spiraled member.

16. The method of claim 1, wherein the tissue removal device comprises an elongate member and a tubular member disposed over the elongate member;
the elongate member having a lengthwise axis and a main body defining a lumen, a proximal portion, a coil member, and a sharp edge, the coil member having a first end and a second end, the first end attached to the proximal portion, the second end free of attachment to the proximal portion, the coil member extending from the first end around a portion of the lengthwise axis in a first direction to the second end, the coil member extending from the first end and away from the lengthwise axis to the second end, the coil member extending away from the first end and away from proximal portion to the second end, the sharp edge defined on the second end of the coil member; and
the tubular member partially disposed over the elongate member and having a proximal end, a distal end, and a main body defining a lumen and a slot, the lumen of the tubular member extending from the proximal end of the tubular member to the distal end of the tubular member, the slot extending from the distal end of the tubular member toward the proximal end of the tubular member, the slot configured to receive a portion of the coil member.

17. The method of claim 1, wherein the tissue removal device comprises an elongate member having a central lengthwise axis, a proximal end, a distal end, and a main body defining a lumen, an inner surface, an outer surface, and a plurality of cutting teeth, each tooth of the plurality of cutting teeth extending from the inner surface toward the central lengthwise of the elongate member and around the entire central lengthwise axis.

18. The method of claim 1, wherein the tissue removal device comprises an elongate member having a lengthwise axis, a proximal end, a distal end, and a main body defining a central shaft, a blunted distal tip, and a plurality of barbs, each barb of the plurality of barbs extending from the central shaft and away from the lengthwise axis of the elongate member.

19. A method of performing treatment under magnetic resonance imaging comprising:
positioning a patient within a magnetic resonance scanner;
scanning a first portion of the patient using the magnetic resonance scanner;
obtaining a magnetic resonance image of the first portion of the patient;
identifying a tissue that has predefined characteristics using the magnetic resonance image;
while the patient is positioned within the magnetic resonance scanner used to scan the first portion of the patient, advancing a medical device into a bodily passage and to the tissue while scanning a second portion of the patient that includes the medical device using the magnetic resonance scanner;
obtaining a magnetic resonance image of the second portion of the patient that includes the medical device;
confirming the position of the medical device within the bodily passage;
advancing a biopsy device through the medical device and to the tissue while scanning a third portion of the patient that includes the biopsy device using the magnetic resonance scanner;
obtaining a magnetic resonance image of the third portion of the patient that includes the biopsy device;
confirming the position of the biopsy device within the bodily passage;
collecting a tissue sample from the tissue using the biopsy device while scanning a fourth portion of the patient that includes the biopsy device and the tissue using the magnetic resonance scanner;
obtaining a magnetic resonance image of the fourth portion of the patient that includes the biopsy device;
confirming the tissue sample has been collected;
withdrawing the biopsy device and the tissue sample through the medical device;
determining whether the tissue sample meets a predefined criterion;
if the tissue sample meets the predefined criterion, advancing an anchor member through the medical device through which the biopsy device was advanced and to the tissue while the patient remains positioned within the magnetic resonance scanner;
securing the anchor member to the tissue to retain the position of the medical device relative to the tissue;
advancing a first inner sheath over the medical device and toward the tissue to dilate the bodily passage;
advancing a second inner sheath over the first inner sheath and toward the tissue to dilate the bodily passage;
advancing an outer sheath over the second inner sheath and toward the tissue to dilate the bodily passage;
removing the anchor member from the tissue;
withdrawing the anchor member from the bodily passage;
withdrawing the medical device from the bodily passage;
withdrawing the first inner sheath from the bodily passage;
withdrawing the second inner sheath from the bodily passage;
advancing a treatment device through the outer sheath and to the tissue;
manipulating the tissue using the treatment device;
withdrawing the treatment device from the outer sheath;
advancing a tissue removal device through the outer sheath and to the tissue, the tissue removal device comprising an elongate member, a plurality of markers disposed on the elongate member, and a tubular member disposed over the elongate member;
the elongate member having a lengthwise axis and a main body defining a lumen, a proximal portion, a coil member, and a sharp edge, the coil member having a first end and a second end, the first end attached to the proximal portion, the second end free of attachment to the proximal portion, the coil member extending from the first end around a portion of the lengthwise axis in a first direction to the second end, the coil member extending from the first end and away from the lengthwise axis to the second end, the coil member extending away from the first end and away from proximal portion to the second end, the sharp edge defined on the second end of the coil member; and
the tubular member partially disposed over the elongate member and having a proximal end, a distal end, and a main body defining a lumen and a slot, the lumen of the tubular member extending from the proximal end of the tubular member to the distal end of the tubular member, the slot extending from the distal end of the tubular member toward the proximal end of the tubular member, the slot configured to receive a portion of the coil member;
manipulating the tissue using the tissue removal device;
withdrawing the tissue removal device from the outer sheath; and
withdrawing the outer sheath from the bodily passage.

20. A tissue removal device useful in performing treatment under magnetic resonance imaging comprising:
an elongate member having a lengthwise axis and a main body defining a lumen, a proximal portion, a coil member, and a sharp edge, the coil member having a first end and a second end, the first end attached to the proximal portion, the second end free of attachment to the proximal portion, the coil member extending from the first end around a portion of the lengthwise axis in a first direction to the second end, the coil member extending from the first end and away from the lengthwise axis to the second end, the coil member extending away from the first end and away from proximal portion to the second end, the sharp edge defined on the second end of the coil member; and
a tubular member partially disposed over the elongate member and having a proximal end, a distal end, and a main body defining a lumen and a slot, the lumen of the tubular member extending from the proximal end of the tubular member to the distal end of the tubular member, the slot extending from the distal end of the tubular member toward the proximal end of the tubular member, the slot configured to receive a portion of the coil member.
